SEQUENCE LISTING (117 sequences)

SEQ ID NO: 1 - protein, 11 aa, synthetic construct
YPREACVQWKV

SEQ ID NO: 2 - protein, 11 aa, synthetic construct
TVAAPCVFIFP

SEQ ID NO: 3 - protein, 11 aa, synthetic construct
QGTKVCIKRTV

SEQ ID NO: 4 - protein, 11 aa, synthetic construct
EPVTVCWNSGA

SEQ ID NO: 5 - protein, 11 aa, synthetic construct
TFPAVCQSSGL

SEQ ID NO: 6 - protein, 11 aa, synthetic construct
VLQSSCLYSLS

SEQ ID NO: 7 - protein, 11 aa, synthetic construct
LQSGNCQESVT

SEQ ID NO: 8 - protein, 11 aa, synthetic construct
YEKHKCYACEV

SEQ ID NO: 9 - protein, 11 aa, synthetic construct
VTHQGCSSPVT

SEQ ID NO: 10 - protein, 11 aa, synthetic construct
TVSSACTKGPS

SEQ ID NO: 11 - protein, 11 aa, synthetic construct
ALTSGCHTFPA

SEQ ID NO: 12 - protein, 11 aa, synthetic construct
GTQTYCCNVNH

SEQ ID NO: 13 - protein, 11 aa, synthetic construct
QLKSGCASVVC

SEQ ID NO: 14 - protein, 11 aa, synthetic construct
SSLGTCTYICN

SEQ ID NO: 15 - protein, 11 aa, synthetic construct
YPSDICVEWES

SEQ ID NO: 16 - protein, 11 aa, synthetic construct
AKVQWCVDNAL

SEQ ID NO: 17 - protein, 11 aa, synthetic construct
KADYECHKVYA

SEQ ID NO: 18 - protein, 11 aa, synthetic construct
TSGGTCALGCL

SEQ ID NO: 19 - protein, 11 aa, synthetic construct
TVSSACTKGPS

SEQ ID NO: 20 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREACVQWKVDNALQSGNSQESVTEQDSKDSTYSLSSTLTLSKADYEKHKVYACEVTHQGLSSPVTKSFNRGEC

SEQ ID NO: 25 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVCIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNCQESVTEQDSKDSTYSLSSTLTLSKADYEKHKVYACEVTHQGLSSPVTKSFNRGEC

SEQ ID NO: 28 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKCYACEVTHQGLSSPVTKSFNRGEC

SEQ ID NO: 29 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGCSSPVTKSFNRGEC

SEQ ID NO: 30 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGCASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWCVDNALQSGNSQESVTEQDSKDSTYSLSSTLTLSKADYEKHKVYACEVTHQGLSSPVTKSFNRGEC

SEQ ID NO: 32 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYECHKVYACEVTHQGLSSPVTKSFNRGEC

SEQ ID NO: 33 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVCWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVCQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSCLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSACTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGCHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYCCNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTCTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 41 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTCALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYECHKVYACEVTHQGLSSPVTKSFNRGEC

SEQ ID NO: 43 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKCYACEVTHQGLSSPVTKSFNRGEC

SEQ ID NO: 44 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGCASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSACTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 23 aa, synthetic construct
DIQMTQSPSSLSASVGDRVTITC

SEQ ID NO: 47 - protein, 11 aa, synthetic construct
RASQDVNTAVA

SEQ ID NO: 48 - protein, 15 aa, synthetic construct
WYQQKPGKAPKLLIY

SEQ ID NO: 49 - protein, 7 aa, synthetic construct
SASFLYS

SEQ ID NO: 50 - protein, 32 aa, synthetic construct
GVPSRFSGSRSGTDFTLTISSLQPEDFATYYC

SEQ ID NO: 51 - protein, 9 aa, synthetic construct
QQHYTTPPT

SEQ ID NO: 52 - protein, 10 aa, synthetic construct
FGQGTKVEIK

SEQ ID NO: 53 - protein, 30 aa, synthetic construct
EVQLVESGGGLVQPGGSLRLSCAASGFNIK

SEQ ID NO: 54 - protein, 5 aa, synthetic construct
DTYIH

SEQ ID NO: 55 - protein, 14 aa, synthetic construct
WVRQAPGKGLEWVA

SEQ ID NO: 56 - protein, 17 aa, synthetic construct
RIYPTNGYTRYADSVKG

SEQ ID NO: 57 - protein, 32 aa, synthetic construct
RFTISADTSKNTAYLQMNSLRAEDTAVYYCSR

SEQ ID NO: 58 - protein, 11 aa, synthetic construct
WGGDGFYAMDY

SEQ ID NO: 59 - protein, 11 aa, synthetic construct
WGQGTLVTVSS

SEQ ID NO: 60 - protein, 23 aa, synthetic construct
DIQLTQSPSSLSASVGDRVSITC

SEQ ID NO: 61 - protein, 11 aa, synthetic construct
KASQDVSIAVA

SEQ ID NO: 62 - protein, 15 aa, synthetic construct
WYQQKPGKAPKLLIY

SEQ ID NO: 63 - protein, 7 aa, synthetic construct
SASYRYT

SEQ ID NO: 64 - protein, 32 aa, synthetic construct
GVPDRFSGSGSGTDFTLTISSLQPEDFAVYYC

SEQ ID NO: 65 - protein, 9 aa, synthetic construct
QQHYITPLT

SEQ ID NO: 66 - protein, 10 aa, synthetic construct
FGAGTKVEIK

SEQ ID NO: 67 - protein, 30 aa, synthetic construct
QVQLQQSGSELKKPGASVKVSCKASGYTFT

SEQ ID NO: 68 - protein, 5 aa, synthetic construct
NYGMN

SEQ ID NO: 69 - protein, 14 aa, synthetic construct
WVKQAPGQGLKWMG

SEQ ID NO: 70 - protein, 17 aa, synthetic construct
WINTYTGEPTYTDDFKG

SEQ ID NO: 71 - protein, 32 aa, synthetic construct
RFAFSLDTSVSTAYLQISSLKADDTAVYFCAR

SEQ ID NO: 72 - protein, 12 aa, synthetic construct
GGFGSSYWYFDV

SEQ ID NO: 73 - protein, 11 aa, synthetic construct
WGQGSLVTVSS

SEQ ID NO: 74 - protein, 23 aa, synthetic construct
DIQMTQSPSSLSASVGDRVTITC

SEQ ID NO: 75 - protein, 11 aa, synthetic construct
KASQDVSTAVA

SEQ ID NO: 76 - protein, 15 aa, synthetic construct
WYQQKPGKAPKLLIY

SEQ ID NO: 77 - protein, 7 aa, synthetic construct
SASYRYT

SEQ ID NO: 78 - protein, 32 aa, synthetic construct
GVPSRFSGSGSGTDFTLTISSLQPEDFAVYYC

SEQ ID NO: 79 - protein, 9 aa, synthetic construct
QQHYITPLT

SEQ ID NO: 80 - protein, 10 aa, synthetic construct
FGQGTKLEIK

SEQ ID NO: 81 - protein, 30 aa, synthetic construct
QVQLVQSGAEVKKPGASVKVSCKASGYTFT

SEQ ID NO: 82 - protein, 5 aa, synthetic construct
TAGMQ

SEQ ID NO: 83 - protein, 14 aa, synthetic construct
WVRQAPGQGLEWMG

SEQ ID NO: 84 - protein, 17 aa, synthetic construct
WINTHSGVPKYAEDFKG

SEQ ID NO: 85 - protein, 32 aa, synthetic construct
RVTISADTSTSTAYLQLSSLKSEDTAVYYCAR

SEQ ID NO: 86 - protein, 12 aa, synthetic construct
SGFGSSYWYFDV

SEQ ID NO: 87 - protein, 11 aa, synthetic construct
WGQGTLVTVSS

SEQ ID NO: 88 - protein, 22 aa, synthetic construct
QSALTQPASVSGSPGQSITISC

SEQ ID NO: 89 - protein, 14 aa, synthetic construct
TGTSSDVGGYNYVS

SEQ ID NO: 90 - protein, 15 aa, synthetic construct
WYQQHPGKAPKLMIY

SEQ ID NO: 91 - protein, 7 aa, synthetic construct
DVSNRPS

SEQ ID NO: 92 - protein, 32 aa, synthetic construct
GVSNRFSGSKSGNTASLTISGLQAEDEADYYC

SEQ ID NO: 93 - protein, 10 aa, synthetic construct
SSYTSSSTRV

SEQ ID NO: 94 - protein, 13 aa, synthetic construct
FGTGTKVTVLGQP

SEQ ID NO: 95 - protein, 30 aa, synthetic construct
EVQLLESGGGLVQPGGSLRLSCAASGFTFS

SEQ ID NO: 96 - protein, 5 aa, synthetic construct
SYIMM

SEQ ID NO: 97 - protein, 14 aa, synthetic construct
WVRQAPGKGLEWVS

SEQ ID NO: 98 - protein, 17 aa, synthetic construct
SIYPSGGITFYADTVKG

SEQ ID NO: 99 - protein, 32 aa, synthetic construct
RFTISRDNSKNTLYLQMNSLRAEDTAVYYCAR

SEQ ID NO: 100 - protein, 11 aa, synthetic construct
IKLGTVTTVDY

SEQ ID NO: 101 - protein, 11 aa, synthetic construct
WGQGTLVTVSS

SEQ ID NO: 102 - protein, 22 aa, synthetic construct
QAVLTQPASLSASPGASASLTC

SEQ ID NO: 103 - protein, 14 aa, synthetic construct
TLRRGINVGAYSIY

SEQ ID NO: 104 - protein, 15 aa, synthetic construct
WYQQKPGSPPQYLLR

SEQ ID NO: 105 - protein, 11 aa, synthetic construct
YKSDSDKQQGS

SEQ ID NO: 106 - protein, 34 aa, synthetic construct
GVSSRFSASKDASANAGILLISGLQSEDEADYYC

SEQ ID NO: 107 - protein, 10 aa, synthetic construct
MIWHSGASAV

SEQ ID NO: 108 - protein, 13 aa, synthetic construct
FGGGTKLTVLGQP

SEQ ID NO: 109 - protein, 30 aa, synthetic construct
EVQLVESGGGLVQPGRSLRLSCAASGFTVS

SEQ ID NO: 110 - protein, 5 aa, synthetic construct
SYWMH

SEQ ID NO: 111 - protein, 14 aa, synthetic construct
WVRQAPGKGLEWVG

SEQ ID NO: 112 - protein, 19 aa, synthetic construct
FIRNKANSGTTEYAASVKG

SEQ ID NO: 113 - protein, 32 aa, synthetic construct
RFTISRDDSKNTLYLQMNSLRAEDTAVYYCAR

SEQ ID NO: 114 - protein, 10 aa, synthetic construct
DRGLRFYFDY

SEQ ID NO: 115 - protein, 11 aa, synthetic construct
WGQGTTVTVSS

SEQ ID NO: 116 - protein, 4 aa, synthetic construct
AAPA

SEQ ID NO: 117 - protein, 4 aa, synthetic construct
AAPV

(19) United States
(12) Patent Application Publication
(10) Pub. No.: US 2025/0000995 A1
(43) Pub. Date: Jan. 2, 2025

(54) TLR AGONIST IMMUNOCONJUGATES WITH CYSTEINE-MUTANT ANTIBODIES, AND USES THEREOF

(71) Applicant: BOLT BIOTHERAPEUTICS, INC., Redwood City, CA (US)

(72) Inventors: Shelley Erin ACKERMAN, Redwood City, CA (US); Michael N. ALONSO, Redwood City, CA (US); Jan GRUNEWALD, Redwood City, CA (US); Romas KUDIRKA, Redwood City, CA (US); Brian SAFINA, Redwood City, CA (US); Ganapathy SARMA, Redwood City, CA (US)

(73) Assignee: BOLT BIOTHERAPEUTICS, INC., Redwood City, CA (US)

(21) Appl. No.: 18/701,881

(22) PCT Filed: Oct. 28, 2022
(86) PCT No.: PCT/US2022/048224

§ 371 Date: Apr. 16, 2024

Related U.S. Application Data
(60) Provisional application No. 63/273,379, filed on Oct. 29, 2021.

(51) Int. Cl. A61K 47/68; A61P 35/00

(52) U.S. Cl. CPC A61K 47/6851; A61K 47/6803; A61K 47/6849; A61K 47/6853; A61K 47/6889; A61P 35/00

(57) ABSTRACT

The invention provides immunoconjugates comprising a cysteine-mutant antibody covalently attached to one or more TLR agonist moieties by a linker. The invention further provides methods of treating cancer with the immunoconjugates.

### CROSS REFERENCE TO RELATED APPLICATIONS

This non-provisional application claims the benefit of priority to U.S. Provisional Application No. 63/273,379, filed 29 Oct. 2021, which is incorporated by reference in its entirety.

### SEQUENCE LISTING

The instant application contains a Sequence Listing which has been submitted in XML format via Patent Center and is hereby incorporated by reference in its entirety. Said XML copy, created on Oct. 28, 2022, is named ST26-17019.019WO1.xml and is 40,668 Bytes in size.

### FIELD OF THE INVENTION

The invention relates generally to an immunoconjugate comprising a cysteine-mutant antibody conjugated to one or more toll-like receptor (TLR) agonist moieties.

### BACKGROUND OF THE INVENTION

New compositions and methods for the delivery of antibodies and immune adjuvants are needed in order to reach inaccessible tumors and/or to expand treatment options for cancer patients and other subjects. The invention provides such compositions and methods.

### SUMMARY OF THE INVENTION

The invention is generally directed to an immunoconjugate comprising a cysteine-mutant antibody covalently attached to one or more TLR agonist moieties by a linker.

Another aspect of the invention is a method of preparing an immunoconjugate by conjugation of one or more TLR agonist-linker compounds with a cysteine-mutant antibody.

Another aspect of the invention is a pharmaceutical composition comprising a therapeutically effective amount of an immunoconjugate comprising a cysteine-mutant antibody covalently attached to one or more TLR agonist moieties by a linker, and one or more pharmaceutically acceptable diluent, vehicle, carrier or excipient.

Another aspect of the invention is a method for treating cancer comprising administering a therapeutically effective amount of an immunoconjugate comprising a cysteine-mutant antibody covalently attached to one or more TLR agonist moieties by a linker.

Another aspect of the invention is a use an immunoconjugate comprising a cysteine-mutant antibody covalently attached to one or more TLR agonist moieties by a linker in the treatment of an illness, in particular cancer.

### BRIEF DESCRIPTION OF THE DRAWINGS

FIG. 1 shows a graph demonstrating IL-12p70 secretion following activation of enriched human cDCs (conventional dendritic cells) freshly isolated from human blood and co-cultured with HCC1954 tumor cells with cysteine-mutant immunoconjugates Lys IC-1 (Table 11), IC-2, IC-3, IC-4, IC-8, IC-10, IC-13, IC-16, IC-17 and IC-18 (Table 10) and unconjugated antibody, trastuzumab. Logarithmic production of IL-12p70 is plotted at increasing concentrations immunoconjugates and trastuzumab

FIG. 2 shows a graph demonstrating IL-12p70 secretion following activation of enriched human cDCs (conventional dendritic cells) freshly isolated from human blood and co-cultured with HCC1954 tumor cells with cysteine-mutant immunoconjugates IC-1, IC-12, IC-6, IC-11, IC-5, IC-9, IC-7, IC-14, and IC-15 (Table 10), Lys IC-1 (Table 11), and unconjugated antibody, trastuzumab. Logarithmic production of IL-12p70 is plotted at increasing concentrations of immunoconjugates and trastuzumab.

FIG. 3 shows a graph demonstrating IL-12p70 secretion following activation of enriched human cDCs (conventional dendritic cells) freshly isolated from human blood and co-cultured with HCC1954 tumor cells with cysteine-mutant, anti-HER2 immunoconjugates IC-8, IC-13, IC-17, and IC-10, and control amide-linked, anti-HER2 conjugate Lys IC-1.

FIG. 4 shows a graph demonstrating IL-12p70 secretion following activation of enriched human cDCs (conventional dendritic cells) freshly isolated from human blood and co-cultured with a modified HCC1954 cell line that overexpresses PD-L1 with cysteine-mutant, anti-PD-L1 immunoconjugates IC-30, IC-31, and control amide-linked, anti-PD-L1 conjugate Lys IC-3.

FIG. 5 shows a graph demonstrating IL-12p70 secretion following activation of enriched human cDCs (conventional dendritic cells) freshly isolated from human blood and co-cultured with HPAF II pancreatic carcinoma tumor cells with cysteine-mutant, anti-TROP2 immunoconjugates, IC-27, IC-28, IC-29, IC-32, and control amide-linked, anti-TROP2 conjugate Lys IC-2.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly available TLR7 sequence, e.g., GenBank accession number AAZ95441 for human TLR8 polypeptide, or GenBank accession number AAK62677 for murine TLR8 polypeptide.

A “TLR agonist” is a compound that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Bispecific” antibodies (bsAbs) are antibodies that bind two distinct epitopes to cancer (Suurs F. V. et al (2019) Pharmacology & Therapeutics 201: 103-119). Bispecific antibodies may engage immune cells to destroy tumor cells, deliver payloads to tumors, and/or block tumor signaling pathways. An antibody that targets a particular antigen includes a bispecific or multispecific antibody with at least one antigen binding region that targets the particular antigen.

In some embodiments, the targeted monoclonal antibody is a bispecific antibody with at least one antigen binding region that targets tumor cells. Such antigens include but are not limited to: mesothelin, prostate specific membrane antigen (PSMA), HER2, TROP2, CEA, EGFR, 5T4, Nectin4, CD19, CD20, CD22, CD30, CD70, B7H3, B7H4 (also known as 08E), protein tyrosine kinase 7 (PTK7), glypican-3, RG1, fucosyl-GMI, CTLA-4, and CD44 (WO 2017/196598).

“Antibody construct” refers to an antibody or a fusion protein comprising (i) an antigen binding domain and (ii) an Fc domain.

The term “immunoconjugate” refers to an antibody construct that is covalently bonded to an adjuvant moiety via a linker. Immunoconjugates allow targeted delivery of an active adjuvant moiety while the target antigen is bound.

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

In some embodiments, the antibody construct is an antigen-binding antibody “fragment,” which comprises at least an antigen-binding region of an antibody, alone or with other components that together constitute the antibody construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab∝ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragment can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

“Cysteine-mutant antibody” is an antibody in which one or more amino acid residues of an antibody are substituted with cysteine residues. A cysteine-mutant antibody may be prepared from the parent antibody by antibody engineering methods (Junutula, et al., (2008b) Nature Biotech., 26(8):925-932; Doman et al. (2009) Blood 114(13):2721-2729; U.S. Pat. Nos. 7,521,541; 7,723,485; US 2012/0121615; WO 2009/052249). Cysteine residues provide for site-specific conjugation of a adjuvant such as a TLR agonist to the antibody through the reactive cysteine thiol groups at the engineered cysteine sites but do not perturb immunoglobulin folding and assembly or alter antigen binding and effector functions. Cysteine-mutant antibodies can be conjugated to the TLR agonist-linker compound with uniform stoichiometry of the immunoconjugate (e.g., up to two TLR agonist moieties per antibody in an antibody that has a single engineered, mutant cysteine site). The TLR agonist-linker compound has a reactive electrophilic group to react specifically with the free cysteine thiol groups of the cysteine-mutant antibody.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natd. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B} (Russell et al., J. Mol Biol., 244: 332-350 (1994).

The “antibody construct” or “binding agent” comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The antibody construct can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally occurring amino acids. For example, “amino acid analogs” can be unnatural amino acids that have the same basic chemical structure as naturally occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C∫CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical. “Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9× to 1.1×, e.g., from 0.95× to 1.05× or from 0.99× to 1.01×. A reference to “about X” or “around X” specifically indicates at least the values X, 0.95×, 0.96×, 0.97×, 0.98×, 0.99×, 1.01×, 1.02×, 1.03×, 1.04×, and 1.05×. Accordingly, “about X” and “around X” are intended to teach and provide written description support for a claim limitation of, e.g., “0.98×.”

### Antibody Targets

In some embodiments, the antibody of an immunoconjugate is capable of binding one or more targets selected from (e.g., specifically binds to a target selected from) 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AMIHR2, ANGPT1, ANGPT2, ANGPTL3, ANGPTL4, ANPEP, APC, APOC1, AR, aromatase, ATX, AX1, AZGP1 (zinc-a-glycoprotein), B7.1, B7.2, B7-H1, BAD, BAFF, BAG1, BAIl, BCR, BCL2, BCL6, BDNF, BLNK, BLR1 (MDR15), BIyS, BMP1, BMP2, BMP13B (GDFIO), BMP4, BMP6, BMP8, BMPRTA, BMPR1B, BMPR2, BPAG1 (plectin), BRCA1, C19orflO (IL27w), C3, C4A, C5, C5R1, CANT1, CAPRIN-1, CASP1, CASP4, CAV1, CCBP2 (D6/JAB61), CCLI1 (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-1), CCL23 (MPIF-I), CCL24 (MPIF-2/eotaxin-2), CCL25 (TECK), CCL26 (eotaxin-3), CCL27 (CTACK/ILC), CCL28, CCL3 (MIP-Ia), CCL4 (MIPIb), CCL5 (RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR⁵ (CMKBR⁵/ChemR13), CCR6 (CMKBR6/CKR-L3/STRL22/DRY6), CCR7 (CKR7/EBI1), CCR8 (CMKBR8/TERI/CKR-L1), CCR9 (GPR-9-6), CCRL1 (VSHK1), CCRL2 (L-CCR), CD164, CD19, CDIC, CD2, CD20, CD21, CD200, CD-22, CD24, CD27, CD28, CD3, CD33, CD35, CD37, CD38, CD3E, CD3G, CD3Z, CD4, CD38, CD40, CD40L, CD44, CD45RB, CD47, CD52, CD69, CD72, CD74, CD79A, CD79B, CD8, CD80, CD81, CD83, CD86, CD137, CD152, CD274, CDH1 (Ecadherin), CDH10, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEBPB, CERI, CHGA, CHGB, Chitinase, CHST10, CKLFSF2, CKLFSF3, CKLFSF4, CKLFSF5, CKLFSF6, CKLFSF7, CKLFSF8, CLDN3, CLDN7 (claudin-7), CLDN18.2 (claudin 18.2), CLN3, CLU (clusterin), CMKLR1, CMKORI (RDC1), CNR1, COL18A1, COLIA1, COL4A3, COL6A1, CR2, Cripto, CRP, CSF1 (M-CSF), CSF2 (GM-CSF), CSF3 (GCSF), CTL8, CTNNB1 (b-catenin), CTSB (cathepsin B), CX3CL1 (SCYD1), CX3CR1 (V28), CXCL1 (GRO1), CXCL10 (IP—IO), CXCL11 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPRA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (Her-2), EREG, ERK8, Estrogen receptor, Earl, ESR2, F3 (TF), FADD, famesyltransferase, FasL, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF). FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FILI (EPSILON), FBL1 (ZETA), FLJ12584, FLJ25530, FLRT1 (fibronectin), FLT1, FLT-3, FOS, FOSL1 (FRA-1), FY (DARC), GABRP (GABAa), GAGEB1, GAGEC1, GALNAC4S-6ST, GATA3, GD2, GDF5, GFI1, GGT1, GM-CSF, GNAS1, GNRH1, GPR2 (CCR10), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIF1A, HIP1, histamine and histamine receptors, HLA-A, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2 (CD121b), IL-IRA, IL-2, IL2RA (CD25), IL2RB (CD122), IL2RG (CD132), IL-4, IL-4R (CD123), IL-5, IL5RA (CD125), IL3RB (CD131), IL-6, IL6RA, (CD126), IR6RB (CD130), IL-7, IL7RA (CD127), IL-8, CXCR1 (IL8RA), CXCR2, (IL8RB/CD128), IL-9, IL9R (CD129), IL-10, IL10RA (CD210), IL10RB (CDW210B), IL-11, IL IRA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, ILIR2, ILIRAP, ILIRAPL1, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4, IL6ST (glycoprotein 130), ILK, INHA, INHBA, INSL3, INSL4, IRAK1, IRAK2, ITGA1, ITGA2, ITGA3, ITGA6 (.alpha.6 integrin), ITGAV, ITGB3, ITGB4 (.beta.4 integrin), JAG1, JAK1, JAK3, JTB, JUN, K6HF, KAIl, KDR, KITLG, KLF5 (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMA5, LEP (leptin), Lingo-p75, Lingo-Troy, LPS, LTA (TNF-b)), LTB, LTB4R (GPR16), LTB4R2, LTBR, MACMARCKS, MAG or OMgp, MAP2K7 (c-Jun), MCP-1, MDK, MIB1, midkine, MIF, MISRII, MJP-2, MK, MKI67 (Ki-67), MMP2, MMP9, MS4A1, MSMB, MT3 (metallothionectin-UI), mTOR, MTSS1, MUC1 (mucin), MYC, MYD88, NCK2, neurocan, Nectin-4, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NRID2, NRIH2, NRIH3, NRIH4, NR112, NR113, NR2C1, NR2C2, NR2E1, NR2E3, NR2F1, NR2F2, NR2F6, NR3C1, NR3C2, NR4A1, NR4A2, NR4A3, NR⁵A1, NR⁵A2, NR6A1, NRP1, NRP2, NT5E, NTN4, ODZI, OPRDI, P2RX7, PAP, PART1, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRKCQ, PRKD1, PRL, PROC, PROK2, PSAP, PSCA, PTAFR, PTEN, PTGS2 (COX-2), PIN, RAC2 (P21Rac2), RANK, RANK ligand, RARB, RGS1, RGS13, RGS3, RNFI1O (ZNF144), Ron, ROBO2, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLC2A2, SLC33A1, SLC43A1, SLIT2, SPP1, SPRR1B (Spr1), ST6GAL1, STAB1, STATE, STEAP, STEAP2, TB4R2, TBX21, TCP1O, TDGF1, TEK, TGFA, TGFB1, TGFB1I1, TGFB2, TGFB3, TGFBI, TGEBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR⁵, TLR6, TLR7, TLR8, TLR9, TLR10, TLR11, TNF, TNF-α, TNFAIP2 (B94), TNFAIP3, TNFRSF11A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFSF11 (TRANCE), TNFSF12 (APO3L), TNFSF13 (April), TNFSF13B, TNSF14 (HVEM-L), TNFRSF14 (HVEM), TNFSF15 (VEGI), TNFSF18, TNFSF4 (OX40 ligand), TNFSF5 (CD40 ligand). TNFSF6 (FasL), TNFSF7 (CD27 ligand), TNFSF8 (CD30 ligand), TNFSF9 (4-1BB ligand), TOLLIP, Toll-like receptors, TOP2A (topoisomerase 1ia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, Wnt-1, XCL1 (tymphotactin), XCL2 (SCM-Ib), XCRI (GPR⁵/CCXCR1), YYI, ZFPM2, CLEC4C (BDCA-2, DLEC, CD303, CLECSF7), CLEC4D (MCL, CLECSF8), CLEC4E (Mincle), CLEC6A (Dectin-2). CLEC5A (MDL-1, CLECSF5), CLECIB (CLEC-2), CLEC9A (DNGR-1), CLEC7A (Dectin-1), PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, TARM1, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC14, SIGLEC15 (CD33L3), SIGLEC16, SIRPB1 (CD172B), TREM1 (CD354), TREM2, and KLRF1 (NKp80).

In some embodiments, the antibody binds to an FcR.gamma-coupled receptor. In some embodiments, the FcR.gamma-coupled receptor is selected from the group consisting of GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, and TARM1.

In some embodiments, the antibody binds to a DAP12-coupled receptor. In some embodiments, the DAP12-coupled receptor is selected from the group consisting of CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44). PILRB, SIGLEC1 (CD169, SN), SIGLEC14, SIGLEC15 (CD33L3), SIGLEC16, SIRPB1 (CD172B), TREM1 (CD354), and TREM2.

In some embodiments, the antibody binds to a hemITAM-bearing receptor. In some embodiments, the hemITAM-bearing receptor is KLRF1 (NKp80).

In some embodiments, the antibody is capable of binding one or more targets selected from CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLECIB (CLEC-2), CLEC9A (DNGR-1), and CLEC7A (Dectin-1). In some embodiments, the antibody is capable of binding CLEC6A (Dectin-2) or CLEC5A. In some embodiments, the antibody is capable of binding CLEC6A (Dectin-2).

In some embodiments, the antibody is capable of binding one or more targets selected from (e.g., specifically binds to a target selected from): ATP5I (Q06185), OAT (P29758), AIFM1 (Q9Z0X1), AOFA (Q64133), MTDC (P18155), CMC1 (Q8BH59), PREP (Q8K411), YMEL1 (088967), LPPRC (Q6PB66), LONM (Q8CGK3), ACON (Q99KI0), QDO1 (Q60597), IDHP (P54071), ALDH2 (P47738), ATPB (P56480), AATM (P05202), TMM93 (Q9CQWO), ERGI3 (Q9CQE7), RTN4 (Q99P72), CL041 (Q8BQR4), ERLN2 (Q8BFZ9), TERA (QO1853), DAD1 (P61804), CALX (P35564), CALU (O35887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH, (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, accession numbers are shown in parentheses.

In some embodiments, the antibody binds to an antigen selected from CDH1, CD19, CD20, CD29, CD30, CD38, CD40, CD47, EpCAM, MUC1, MUC16, EGFR, Her2, SLAMF7, and gp75. In some embodiments, the antigen is selected from CD19, CD20, CD47, EpCAM, MUC1, MUC16, EGFR, and Her2. In some embodiments, the antibody binds to an antigen selected from the Tn antigen and the Thomsen-Friedenreich antigen.

In some embodiments, the antibody or Fc fusion protein is selected from: abagovomab, abatacept (also known as ORENCIA®), abciximab (also known as REOPRO®), c7E3 Fab), adalimumab (also known as HUMIRA®), adecatumumab, alemtuzumab (also known as CAMPATH®), MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCINT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN®), exbivirumab, fanolesomab (also known as NEUTROSPEC®), faralimomab, felvizumab, fontolizumab (also known as HUZAF®), galiximab, gantenerumab, gavilimomab (also known as ABXCBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imeiromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-STO1), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA®), metelimumab, milatuzumab, minretumomab, mitumomab, morolimumab, motavizwnab (also known as NUMAX®), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemtumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. In some embodiments, the antibody is rituximab.

### Cysteine-Mutant Antibodies

The immunoconjugate of the invention comprises a cysteine-mutant antibody.

Exemplary embodiments of immunoconjugates comprise a cysteine-mutant antibody with a cysteine mutation selected from the group consisting of: K145C, S114C, E105C, S157C, L174C, G178C, S159C, V191C, L201C, S119C, V167C, I199C, T129C, Q196C, A378C, K149C, K188C, and A140C, numbered according to the EU format.

In some embodiments the cysteine-mutant antibody comprises a substitution of one or more amino acids with cysteine selected from certain positions of a heavy chain of the antibody or antibody fragment, including but not limited to those in Tables 3, 4, 7 and wherein the positions are numbered according to the EU format.

In some embodiments a cysteine-mutant antibody comprises a substitution of one or more amino acids with cysteine on its constant region selected from certain positions of a light chain of the antibody or antibody fragment, including but not limited to those in Tables 1, 2, 5, 6, wherein the positions are numbered according to the EU system, and wherein the light chain is a human kappa light chain.

In one embodiment, the hinge region of CH1 is modified such that the number of cysteine residues in the hinge region is altered; increased or decreased (U.S. Pat. No. 5,677,425). The number of cysteine residues in the hinge region of CH1 may be altered to, for example, facilitate assembly of the light and heavy chains or to increase or decrease the stability of the antibody. Sites for cysteine substitution are selected to provide stable and homogeneous conjugates. A modified antibody or fragment can have two or more cysteine substitutions, and these substitutions can be used in combination with other antibody modification and conjugation methods as described herein. Methods for inserting cysteine at specific locations of an antibody are known in the art, see, e.g., Lyons et al, (1990) Protein Eng., 3:703-708, WO 2011/005481, WO2014/124316, WO 2015/138615.

Included in the scope of the embodiments of the invention are functional variants of the cysteine-mutant antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing, for example but not limited to, PD-L1, HER2, CEA or TROP2, to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fc engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, US 2016/0145350; U.S. Pat. Nos. 7,416,726; and 5,624,821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the Fc region of the antibodies of the immunoconjugates are modified to have an altered glycosylation pattern of the Fc region compared to the native non-modified Fc region. Human immunoglobulin is glycosylated at the Asn297 residue in the C72 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while afucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, US 2007/0014795, and US 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1 antibody constructs, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering a TLR agonist payload to a cell expressing PD-L1 comprising administering to the cell, or mammal comprising the cell, an immunoconjugate comprising a cysteine-mutant, anti-PD-L1 antibody covalently attached to a linker which is covalently attached to one or more TLR agonist moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 antibody comprising an immunoglobulin heavy chain variable region polypeptide and an immunoglobulin light chain variable region polypeptide. The PD-L1 antibody specifically binds PD-L1. The binding specificity of the antibody allows for targeting PD-L1 expressing cells, for instance, to deliver therapeutic payloads to such cells. In some embodiments, the PD-L1 antibody binds to human PD-L1. However, antibodies that bind to any PD-L1 fragment, homolog or paralog also are encompassed.

In some embodiments, the PD-L1 antibody binds PD-L1 without substantially inhibiting or preventing PD-L1 from binding to its receptor, PD-1. However, in other embodiments, the PD-L1 antibody can completely or partially block (inhibit or prevent) binding of PD-L1 to its receptor, PD-1, such that the antibody can be used to inhibit PD-L1/PD-1 signaling (e.g., for therapeutic purposes). The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1 antibody can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

The PD-L1 antibody can be internalizing, as described in WO 2021/150701 and incorporated by reference herein, or the PD-L1 antibody can be non-internalizing, as described in WO 2021/150702 and incorporated by reference herein.

In an exemplary embodiment, the immunoconjugates of the invention comprise an antibody construct that comprises an antigen binding domain that specifically recognizes and binds HER2.

In certain embodiments, immunoconjugates of the invention comprise a cysteine-mutant, anti-HER2 antibody such as those prepared by the methods of Example 201. In one embodiment of the invention, an anti-HER2 antibody of an immunoconjugate of the invention comprises a cysteine-mutant version of a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

Margetuximab (also called MGAH22) is another anti-HER2 monoclonal antibody. The Fc region of margetuximab is optimized for increased binding to the activating Fc gamma Rs but decreased binding to the inhibitory Fc.gamma.Rs on immune effector cells. Margetuximab is approved by the FDA for treatment of patients with relapsed or refractory advanced breast cancer whose tumors express HER2 at the 2+ level by immunohistochemistry and lack evidence of HER2 gene amplification by FISH.

HT-19 is another anti-HER2 monoclonal antibody that binds to an epitope in human HER2 distinct from the epitope of trastuzumab or pertuzumab. HT-19 was shown to inhibit HER2 signaling comparable to trastuzumab and enhance HER2 degradation in combination with trastuzumab and pertuzumab (Bergstrom D. A. et al., (2015) Cancer Res.; 75:LB-231).

In an embodiment of the invention, an immunoconjugate comprises a cysteine-mutant, antibody with a light chain sequence selected from Table 1.

TABLE 1
Cysteine mutation light chain sequences

| Sequence: | mutant site | SEQ ID NO: |
|---|---|---|
| YPREACVQWKV | LC K145C | 1 |
| TVAAPCVFIFP | LC S114C | 2 |
| QGTKVCIKRTV | LC E105C | 3 |
| LQSGNCQESVT | LC S159C | 7 |
| YEKHKCYACEV | LC V191C | 8 |
| VTHQGCSSPVT | LC L201C | 9 |
| QLKSGCASVVC | LC T129C | 13 |
| AKVQWCVDNAL | LC K149C | 16 |
| KADYECHKVYA | LC K188C | 17 |

In an embodiment of the invention, the cysteine-mutant, HER2-targeting antibody comprises the heavy chain (HC) of SEQ ID NO: 20.

SEQ ID NO. 20
EVQLVESGGGLVQPGGSLRLSCAASGFNIKDTYIHWVRQAPGKGLE
WVARIYPTNGYTRYADSVKGRFTISADTSKNTAYLQMNSLRAEDT
AVYYCSRWGGDGFYAMDYWGQGTLVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK

In an embodiment of the invention, the light chain (LC) of a cysteine-mutant, HER2-targeting antibody is selected from SEQ ID NO: 24, 25, 26, 27, 28, 29, 30, 31, and 32 of Table 2.

TABLE 2
Anti-HER2, cysteine mutation light
chain (LC) sequences

| | Cys Mutant | SEQ ID |
|---|---|---|
| Light chain | site | NO: |
| 214aa | LC K145C | 24 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREACVQWKV | | |
| DNALQSGNSQESVTEQDSKDSTYSL | | |
| SSTLTLSKADYEKHKVYACEVTHQG | | |
| LSSPVTKSFNRGEC | | |
| 214aa | LC S114C | 25 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPCVFIFPPSDEQL | | |
| KSGTASVVCLLNNFYPREAKVQWKV | | |
| DNALQSGNSQESVTEQDSKDSTYSL | | |
| SSTLTLSKADYEKHKVYACEVTHQG | | |
| LSSPVTKSFNRGEC | | |
| 214aa | LC E105C | 26 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVCIKRTVAAPSVFIFPPSDEQL | | |
| KSGTASVVCLLNNFYPREAKVQWKV | | |
| DNALQSGNSQESVTEQDSKDSTYSL | | |
| SSTLTLSKADYEKHKVYACEVTHQG | | |
| LSSPVTKSFNRGEC | | |
| 214aa | LC S159C | 27 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREAKVQWKV | | |
| DNALQSGNCQESVTEQDSKDSTYSL | | |
| SSTLTLSKADYEKHKVYACEVTHQG | | |
| LSSPVTKSFNRGEC | | |
| 214aa | LC V191C | 28 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREAKVQWKV | | |
| DNALQSGNSQESVTEQDSKDSTYSL | | |
| SSTLTLSKADYEKHKCYACEVTHQG | | |
| LSSPVTKSFNRGEC | | |
| 214aa | LC L201C | 29 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREAKVQWKV | | |
| DNALQSGNSQESVTEQDSKDSTYSL | | |
| SSTLTLSKADYEKHKVYACEVTHQG | | |
| CSSPVTKSFNRGEC | | |
| CDS00372-214aa | LC T129C | 30 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGCASVVCLLNNFYPREAKVQWKV | | |
| DNALQSGNSQESVTEQDSKDSTYSL | | |
| SSTLTLSKADYEKHKVYACEVTHQG | | |
| LSSPVTKSFNRGEC | | |
| 214aa | LC K149C | 31 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREAKVQWCV | | |
| DNALQSGNSQESVTEQDSKDSTYSL | | |
| SSTLTLSKADYEKHKVYACEVTHQG | | |
| LSSPVTKSFNRGEC | | |
| 214aa | LC K188C | 32 |
| DIQMTQSPSSLSASVGDRVTITCRA | | |
| SQDVNTAVAWYQQKPGKAPKLLIYS | | |
| ASFLYSGVPSRFSGSRSGTDFTLTI | | |
| SSLQPEDFATYYCQQHYTTPPTFGQ | | |
| GTKVEIKRTVAAPSVFIFPPSDEQL | | |
| KSGTASVVCLLNNFYPREAKVQWKV | | |
| DNALQSGNSQESVTEQDSKDSTYSL | | |
| SSTLTLSKADYECHKVYACEVTHQG | | |
| LSSPVTKSFNRGEC | | |

In an embodiment of the invention, an immunoconjugate comprises a cysteine-mutant, antibody with a heavy chain sequence selected from Table 3.

TABLE 3
Cysteine mutation heavy chain sequences

| | | |
|---|---|---|
| EPVTVCWNSGA | HC S157C | 4 |
| TFPAVCQSSGL | HC L174C | 5 |
| VLQSSCLYSLS | HC G178C | 6 |
| TVSSACTKGPS | HC S119C | 10 |
| ALTSGCHTFPA | HC V167C | 11 |
| GTQTYCCNVNH | HC I199C | 12 |
| SSLGTCTYICN | HC Q196C | 14 |
| YPSDICVEWES | HC A378C | 15 |
| TSGGTCALGCL | HC A140C | 18 |

In an embodiment of the invention, the light chain of a cysteine-mutant, HER2-targeting antibody has the sequence of SEQ TD NO:21.

SEQ ID NO. 21
DIQMTQSPSSLSASVGDRVTITCRASQDVNTAVAWYQQKPGKAPKL
LIYSASFLYSGVPSRFSGSRSGTDFTLTISSLQPEDFATYYCQQH
YTTPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC

In an embodiment of the invention, the heavy chain (HC) of a cysteine-mutant, HTER2-targeting antibody is selected from SEQ TD NO: 33, 34, 35, 36, 37, 38, 39, 40, and 41 of Table 4.

TABLE 4
Anti-HER2, cysteine mutation heavy
chain (HC) sequences

| | Cys Mutant | SEQ ID |
|---|---|---|
| Heavy chain | site | NO: |
| 450aa | HC S157C | 33 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVCWNSGALTSGVHTFPA | | |
| VLQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HCL174C | 34 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVSWNSGALTSGVHTFPA | | |
| VCQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HC G178C | 35 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVSWNSGALTSGVHTFPA | | |
| VLQSSCLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HC S119C | 36 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSACTKG | | |
| PSVFPLAPSSKSTSGGTAALGCLVK | | |
| DYFPEPVTVSWNSGALTSGVHTFPA | | |
| VLQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HC V167C | 37 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVSWNSGALTSGCHTFPA | | |
| VLQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HC 1199C | 38 |
| EVQLVESGGGLVQPGGSLRLSCAAS | | |
| GFNIKDTYIHWVRQAPGKGLEI199 | | |
| CWVARIYPTNGYTRYADSVKGRFTI | | |
| SADTSKNTAYLQMNSLRAEDTAVYY | | |
| CSRWGGDGFYAMDYWGQGTLVTVSS | | |
| ASTKGPSVFPLAPSSKSTSGGTAAL | | |
| GCLVKDYFPEPVTVSWNSGALTSGV | | |
| HTFPAVLQSSGLYSLSSVVTVPSSS | | |
| LGTQTYCCNVNHKPSNTKVDKKVEP | | |
| KSCDKTHTCPPCPAPELLGGPSVFL | | |
| FPPKPKDTLMISRTPEVTCVVVDVS | | |
| HEDPEVKFNWYVDGVEVHNAKTKPR | | |
| EEQYNSTYRVVSVLTVLHQDWLNGK | | |
| EYKCKVSNKALPAPIEKTISKAKGQ | | |
| PREPQVYTLPPSREEMTKNQVSLTC | | |
| LVKGFYPSDIAVEWESNGQPENNYK | | |
| TTPPVLDSDGSFFLYSKLTVDKSRW | | |
| QQGNVFSCSVMHEALHNHYTQKSLS | | |
| LSPGK | | |
| SEQ ID NO: 38 | | |
| 450aa | HC Q196C | 39 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVSWNSGALTSGVHTFPA | | |
| VLQSSGLYSLSSVVTVPSSSLGTCT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |
| 450aa | HC A378C | 40 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTAALGCLVK | | |
| DYFPEPVTVSWNSGALTSGVHTFPA | | |
| VLQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDICVEWESNGQPENNYKTTPPV | | |
| LDSDGSFFLYSKLTVDKSRWQQGNV | | |
| FSCSVMHEALHNHYTQKSLSLSPGK | | |
| 450aa | HC A140C | 41 |
| EVQLVESGGGLVQPGGSLRLSCAAS | | |
| GFNIKDTYIHWVRQAPGKGLEWVAR | | |
| IYPTNGYTRYADSVKGRFTISADTS | | |
| KNTAYLQMNSLRAEDTAVYYCSRWG | | |
| GDGFYAMDYWGQGTLVTVSSASTKG | | |
| PSVFPLAPSSKSTSGGTCALGCLVK | | |
| DYFPEPVTVSWNSGALTSGVHTFPA | | |
| VLQSSGLYSLSSVVTVPSSSLGTQT | | |
| YICNVNHKPSNTKVDKKVEPKSCDK | | |
| THTCPPCPAPELLGGPSVFLFPPKP | | |
| KDTLMISRTPEVTCVVVDVSHEDPE | | |
| VKFNWYVDGVEVHNAKTKPREEQYN | | |
| STYRVVSVLTVLHQDWLNGKEYKCK | | |
| VSNKALPAPIEKTISKAKGQPREPQ | | |
| VYTLPPSREEMTKNQVSLTCLVKGF | | |
| YPSDIAVEWESNGQPENNYKTTPPV | | |
| LDSDGSFFLYSKLTVDKSRWQQGNV | | |
| FSCSVMHEALHNHYTQKSLSLSPGK | | |

In an exemplary embodiment, the immunoconjugates of the invention comprise a cysteine-mutant antibody construct that comprises an antigen binding domain that specifically recognizes and binds Caprin-1 (Ellis J A, Luzio J P (1995) J Biol Chem. 270(35):20717-23; Wang B, et al (2005) J Immunol. 175 (7):4274-82; Solomon S, et al (2007) Mol CellBiol. 27(6):2324-42). Caprin-1 is also known as GPIAP1, GPIP137, GRIP137, MI1S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z—S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise a cysteine-mutant antibody construct that comprises an antigen binding domain that specifically recognizes and binds CEA. Carcinoembryonic antigen-related cell adhesion molecule 5 (CEACAM5) also known as CD66e (Cluster of Differentiation 66e), is a member of the carcinoembryonic antigen (CEA) gene family.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having anti-apoptosis functions. CEA is also used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160). In an embodiment of the invention, the CEA-targeting antibody construct or antigen binding domain comprises the Variable light chain (VL kappa) of hMN-14/labetuzumab as disclosed in U.S. Pat. No. 6,676,924, which is incorporated by reference herein for this purpose.

In an exemplary embodiment, the immunoconjugates of the invention comprise a cysteine-mutant antibody construct that comprises an antigen binding domain that specifically recognizes and binds TROP2. Tumor-associated calcium signal transducer 2 (TROP-2) is a transmembrane glycoprotein encoded by the TACSTD2 gene (Linnernbach A J, et al (1993)Mol Cell Biol. 13(3): 1507-15; Calabrese G, et al (2001) Cytogenet Cell Genet. 92(1-2): 164-5). TROP2 is an intracellular calcium signal transducer that is differentially expressed in many cancers and signals cells for self-renewal, proliferation, invasion, and survival. TROP2 is considered a stem cell marker and is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of TROP2 is of prognostic significance. Several ligands have been proposed that interact with TROP2. TROP2 signals the cells via different pathways and it is transcriptionally regulated by a complex network of several transcription factors.

Human TROP2 (TACSTD2: tumor-associated calcium signal transducer 2, GA733-1, EGP-1, MIS1; hereinafter, referred to as hTROP2) is a single-pass transmembrane type 1 cell membrane protein consisting of 323 amino acid residues. While the presence of a cell membrane protein involved in immune resistance, which is common to human trophoblasts and cancer cells (Faulk W P, et al., Proc. Natl. Acad. Sci. 75(4):1947-1951 (1978)), has previously been suggested, an antigen molecule recognized by a monoclonal antibody against a cell membrane protein in a human choriocarcinoma cell line was identified and designated as TROP2 as one of the molecules expressed in human trophoblasts (Lipinski M, et al., Proc. Natl. Acad. Sci. 78(8), 5147-5150 (1981)). This molecule was also designated as tumor antigen GA733-1 recognized by a mouse monoclonal antibody GA733 (Linnenbach A J, et al., Proc. Natl. Acad. Sci. 86(1), 27-31 (1989)) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the TROP2 gene was cloned, and all of these molecules were confirmed to be identical molecules (Fornaro M, et al., Int. J. Cancer, 62(5), 610-618 (1995)). The DNA sequence and amino acid sequence of hTROP2 are available on a public database and can be referred to, for example, under Accession Nos. NM_002353 and NP_002344 (NCBI).

In response to such information suggesting the association with cancer, a plurality of anti-hTROP2 antibodies have been established so far and studied for their antitumor effects. Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; U.S. Pat. No. 7,999,083). However, the strength or coverage of their activity is still insufficient, and there are unsatisfied medical needs for hTROP2 as a therapeutic target.

TROP2 expression in cancer cells has been correlated with drug resistance. Several strategies target TROP2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of TROP2 as both a prognostic biomarker and as a therapeutic target to reverse resistance.

Sacituzumab govitecan (TRODELVY®, Immunomedics, IMMU-132), an antibody-drug conjugate comprising a TROP2-directed antibody linked to a topoisomerase inhibitor drug, is indicated for the treatment of metastatic triple-negative breast cancer (mTNBC) in adult patients that have received at least two prior therapies. The TROP2 antibody in sacituzumab govitecan is conjugated to SN-38, the active metabolite of irinotecan (US 2016/0297890; WO 2015/098099).

In an embodiment of the invention, the TROP2-targeting antibody construct or antigen binding domain comprises the light chain CDR (complementarity determining region) of hRS7 (humanized RS7), (U.S. Pat. No. 7,238,785, incorporated by reference herein).

In an embodiment of the invention, an immunoconjugate comprises a cysteine-mutant, antibody with a light chain sequence selected from Table 5.

TABLE 5
Cysteine mutation light chain sequences

| Sequence: | mutant site | SEQ ID NO: |
|---|---|---|
| KADYECHKVYA | LC K188C | 17 |
| YEKHKCYACEV | LC V191C | 8 |
| QLKSGCASVVC | LC T129C | 13 |

In an embodiment of the invention, a cysteine-mutant, TROP2-targeting antibody comprises the heavy chain (HC) of SEQ TD NO: 22.

SEQ ID NO: 22
QVQLQQSGSELKKPGASVKVSCKASGYTFTNYGMNWVKQAPGQGLK
WMGWINTYTGEPTYTDDFKGRFAFSLDTSVSTAYLQISSLKADDT
AVYFCARGGFGSSYWYFDVWGQGSLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKRVEPKSCDK
THTCPPCPAPELLGGPSVFLFPPKPKDTLMISRTPEVTCVVVDVS
HEDPEVKFNWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK

In an embodiment of the invention, the light chain (LC) of a TROP2-targeting antibody is selected from SEQ ID NO: 42, 43, and 44 of Table 6.

TABLE 6
Anti-TROP2, cysteine mutation light
chain (LC) sequences

| | Cys | SEQ |
|---|---|---|
| | Mutant | ID |
| Heavy chain | site | NO: |
| DIQLTQSPSSLSASVGDRVSITCKASQDVS | LC K188C | 42 |
| IAVAWYQQKPGKAPKLLIYSASYRYTGVPD | | |
| RFSGSGSGTDFTLTISSLQPEDFAVYYCQQ | | |
| HYITPLTFGAGTKVEIKRTVAAPSVFIFPP | | |
| SDEQLKSGTASVVCLLNNFYPREAKVQWKV | | |
| DNALQSGNSQESVTEQDSKDSTYSLSSTLT | | |
| LSKADYECHKVYACEVTHQGLSSPVTKSFN | | |
| RGEC | | |
| DIQLTQSPSSLSASVGDRVSITCKASQDVS | LC V191C | 43 |
| IAVAWYQQKPGKAPKLLIYSASYRYTGVPD | | |
| RFSGSGSGTDFTLTISSLQPEDFAVYYCQQ | | |
| HYITPLTFGAGTKVEIKRTVAAPSVFIFPP | | |
| SDEQLKSGTASVVCLLNNFYPREAKVQWKV | | |
| DNALQSGNSQESVTEQDSKDSTYSLSSTLT | | |
| LSKADYEKHKCYACEVTHQGLSSPVTKSFN | | |
| RGEC | | |
| DIQLTQSPSSLSASVGDRVSITCKASQDVS | LC T129C | 44 |
| IAVAWYQQKPGKAPKLLIYSASYRYTGVPD | | |
| RFSGSGSGTDFTLTISSLQPEDFAVYYCQQ | | |
| HYITPLTFGAGTKVEIKRTVAAPSVFIFPP | | |
| SDEQLKSGCASVVCLLNNFYPREAKVQWKV | | |
| DNALQSGNSQESVTEQDSKDSTYSLSSTLT | | |
| LSKADYEKHKVYACEVTHQGLSSPVTKSFN | | |
| RGEC | | |

In an embodiment of the invention, an immunoconjugate comprises a cysteine-mutant, antibody with a heavy chain sequence selected from Table 7.

TABLE 7
Cysteine mutation heavy chain
sequences

| | Cys mutant | |
|---|---|---|
| Sequence: | site | SEQ ID NO: |
| TVSSACTKGPS | HC | 19 |
| | S119C | |

In an embodiment of the invention, the light chain (LC) of a cysteine-mutant, TROP2-targeting antibody has the sequence of SEQ ID NO:23.

SEQ ID NO: 23
DIQLTQSPSSLSASVGDRVSITCKASQDVSIAVAWYQQKPGKAPKL
LIYSASYRYTGVPDRFSGSGSGTDFTLTISSLQPEDFAVYYCQQH
YITPLTFGAGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC

In an embodiment of the invention, the heavy chain (HC) of a cysteine-mutant, TROP2-targeting antibody has the sequence of SEQ ID NO:45.

SEQ ID NO: 45
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSACTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPGK

In an embodiment of the invention, the cysteine-mutant HER2-targeting antibody construct or antigen binding domain comprises the light chain CDR (complementarity determining region) or light chain framework (LFR) sequences selected from SEQ ID NO. 46-52.

| | | Residues | | SEQ ID |
|---|---|---|---|---|
| Region | Sequence | (Kabat) | Length | NO. |
| LFR1 | DIQMTQSPSSLSASVGDRVTITC | 1-23 | 23 | 46 |
| CDR-L1 | RASQDVNTAVA | 24-34 | 11 | 47 |
| LFR2 | WYQQKPGKAPKLLIY | 35-49 | 15 | 48 |
| CDR-L2 | SASFLYS | 50-56 | 7 | 49 |
| LFR3 | GVPSRFSGSRSGTDFTLTISSLQPEDFATYYC | 57-88 | 32 | 50 |
| CDR-L3 | QQHYTTPPT | 89-97 | 9 | 51 |
| LFR4 | FGQGTKVEIK | 98-107 | 10 | 52 |

In an embodiment of the invention, the cysteine-mutant HER2-targeting antibody construct or antigen binding domain comprises the heavy chain CDR (complementarity determining region) or heavy chain framework (HFR) sequences selected from SEQ TD NO. 53-59.

| | | Residues | | SEQ ID |
|---|---|---|---|---|
| Region | Sequence | (Kabat) | Length | NO. |
| HFR1 | EVQLVESGGGLVQPGGSLRLSCAASGFNIK | 1-30 | 30 | 53 |
| CDR-H1 | DTYIH | 31-35 | 5 | 54 |
| HFR2 | WVRQAPGKGLEWVA | 36-49 | 14 | 55 |
| CDR-H2 | RIYPTNGYTRYADSVKG | 50-66 | 17 | 56 |
| HFR3 | RFTISADTSKNTAYLQMNSLRAEDTAVYYCSR | 67-98 | 32 | 57 |
| CDR-H3 | WGGDGFYAMDY | 99-109 | 11 | 58 |
| HFR4 | WGQGTLVTVSS | 110-120 | 11 | 59 |

In an embodiment of the invention, the cysteine-mutant TROP2-targeting antibody construct or antigen binding domain comprises the light chain CDR (complementarity determining region) or light chain framework (LFR) sequences selected from SEQ TD NO. 60-66.

| | | Residues | | SEQ ID |
|---|---|---|---|---|
| Region | Sequence | (Kabat) | Length | NO. |
| LFR1 | DIQLTQSPSSLSASVGDRVSITC | 1-23 | 23 | 60 |
| CDR-L1 | KASQDVSIAVA | 24-34 | 11 | 61 |
| LFR2 | WYQQKPGKAPKLLIY | 35-49 | 15 | 62 |
| CDR-L2 | SASYRYT | 50-56 | 7 | 63 |
| LFR3 | GVPDRFSGSGSGTDFTLTISSLQPEDFAVYYC | 57-88 | 32 | 64 |
| CDR-L3 | QQHYITPLT | 89-97 | 9 | 65 |
| LFR4 | FGAGTKVEIK | 98-107 | 10 | 66 |

In an embodiment of the invention, the cysteine-mutant TROP2-targeting antibody construct or antigen binding domain comprises the heavy chain CDR (complementarity determining region) or heavy chain framework (HFR) sequences selected from SEQ TD NO. 67-73.

| | | Residues | | SEQ ID |
|---|---|---|---|---|
| Region | Sequence | (Kabat) | Length | NO. |
| HFR1 | QVQLQQSGSELKKPGASVKVSCKASGYTFT | 1-30 | 30 | 67 |
| CDR-H1 | NYGMN | 31-35 | 5 | 68 |
| HFR2 | WVKQAPGQGLKWMG | 36-49 | 14 | 69 |
| CDR-H2 | WINTYTGEPTYTDDFKG | 50-66 | 17 | 70 |
| HFR3 | RFAFSLDTSVSTAYLQISSLKADDTAVYFCAR | 67-98 | 32 | 71 |
| CDR-H3 | GGFGSSYWYFDV | 99-110 | 12 | 72 |
| HFR4 | WGQGSLVTVSS | 111-121 | 11 | 73 |

In an embodiment of the invention, the cysteine-mutant TROP2-targeting antibody construct or antigen binding domain comprises the light chain CDR (complementarity determining region) or light chain framework (LFR) sequences selected from SEQ TD NO. 74-80.

| | | Residues | | SEQ ID |
|---|---|---|---|---|
| Region | Sequence | (Kabat) | Length | NO. |
| LFR1 | DIQMTQSPSSLSASVGDRVTITC | 1-23 | 23 | 74 |
| CDR-L1 | KASQDVSTAVA | 24-34 | 11 | 75 |
| LFR2 | WYQQKPGKAPKLLIY | 35-49 | 15 | 76 |
| CDR-L2 | SASYRYT | 50-56 | 7 | 77 |
| LFR3 | GVPSRFSGSGSGTDFTLTISSLQPEDFAVYYC | 57-88 | 32 | 78 |
| CDR-L3 | QQHYITPLT | 89-97 | 9 | 79 |
| LFR4 | FGQGTKLEIK | 98-107 | 10 | 80 |

In an embodiment of the invention, the cysteine-mutant TROP2-targeting antibody construct or antigen binding domain comprises the heavy chain CDR (complementarity determining region) or heavy chain framework (HFR) sequences selected from SEQ TD NO. 81-87.

| | | Residues | | SEQ ID |
|---|---|---|---|---|
| Region | Sequence | (Kabat) | Length | NO. |
| HFR1 | QVQLVQSGAEVKKPGASVKVSCKASGYTFT | 1-30 | 30 | 81 |
| CDR-H1 | TAGMQ | 31-35 | 5 | 82 |
| HFR2 | WVRQAPGQGLEWMG | 36-49 | 14 | 83 |
| CDR-H2 | WINTHSGVPKYAEDFKG | 50-66 | 17 | 84 |
| HFR3 | RVTISADTSTSTAYLQLSSLKSEDTAVYYCAR | 67-98 | 32 | 85 |
| CDR-H3 | SGFGSSYWYFDV | 99-110 | 12 | 86 |
| HFR4 | WGQGTLVTVSS | 111-121 | 11 | 87 |

In an embodiment of the invention, the cysteine-mutant PD-L1-targeting antibody construct or antigen binding domain comprises the light chain CDR (complementarity determining region) or light chain framework (LFR) sequences selected from SEQ TD NO. 88-94.

| | | Residues | | SEQ ID |
|---|---|---|---|---|
| Region | Sequence | (Kabat) | Length | NO. |
| LFR1 | QSALTQPASVSGSPGQSITISC | 1-22 | 22 | 88 |
| CDR-L1 | TGTSSDVGGYNYVS | 23-36 | 14 | 89 |
| LFR2 | WYQQHPGKAPKLMIY | 37-51 | 15 | 90 |
| CDR-L2 | DVSNRPS | 52-58 | 7 | 91 |
| LFR3 | GVSNRFSGSKSGNTASLTISGLQAEDEADYYC | 59-90 | 32 | 92 |
| CDR-L3 | SSYTSSSTRV | 91-100 | 10 | 93 |
| LFR4 | FGTGTKVTVLGQP | 101-113 | 13 | 94 |

In an embodiment of the invention, the cysteine-mutant PD-L1-targeting antibody construct or antigen binding domain comprises the heavy chain CDR (complementarity determining region) or heavy chain framework (HFR) sequences selected from SEQ TD NO. 95-101.

| | | Residues | | SEQ ID |
|---|---|---|---|---|
| Region | Sequence | (Kabat) | Length | NO. |
| HFR1 | EVQLLESGGGLVQPGGSLRLSCAASGFTFS | 1-30 | 30 | 95 |
| CDR-H1 | SYIMM | 31-35 | 5 | 96 |
| HFR2 | WVRQAPGKGLEWVS | 36-49 | 14 | 97 |
| CDR-H2 | SIYPSGGITFYADTVKG | 50-66 | 17 | 98 |
| HFR3 | RFTISRDNSKNTLYLQMNSLRAEDTAVYYCAR | 67-98 | 32 | 99 |
| CDR-H3 | IKLGTVTTVDY | 99-109 | 11 | 100 |
| HFR4 | WGQGTLVTVSS | 110-120 | 11 | 101 |

In an embodiment of the invention, the cysteine-mutant CEA-targeting antibody construct or antigen binding domain comprises the light chain CDR (complementarity determining region) or light chain framework (LFR) sequences selected from SEQ TD NO. 102-108.

| | | Residues | | SEQ ID |
|---|---|---|---|---|
| Region | Sequence | (Kabat) | Length | NO. |
| LFR1 | QAVLTQPASLSASPGASASLTC | 1-22 | 22 | 102 |
| CDR-L1 | TLRRGINVGAYSIY | 23-36 | 14 | 103 |
| LFR2 | WYQQKPGSPPQYLLR | 37-51 | 15 | 104 |
| CDR-L2 | YKSDSDKQQGS | 52-62 | 11 | 105 |
| LFR3 | GVSSRFSASKDASANAGILLISGLQSEDEADYYC | 63-96 | 34 | 106 |
| CDR-L3 | MIWHSGASAV | 97-106 | 10 | 107 |
| LFR4 | FGGGTKLTVLGQP | 107-119 | 13 | 108 |

In an embodiment of the invention, the cysteine-mutant CEA-targeting antibody construct or antigen binding domain comprises the heavy chain CDR (complementarity determining region) or heavy chain framework (HFR) sequences selected from SEQ TD NO. 109-115.

| | | Residues | | SEQ ID |
|---|---|---|---|---|
| Region | Sequence | (Kabat) | Length | NO. |
| HFR1 | EVQLVESGGGLVQPGRSLRLSCAASGFTVS | 1-30 | 30 | 109 |
| CDR-H1 | SYWMH | 31-35 | 5 | 110 |
| HFR2 | WVRQAPGKGLEWVG | 36-49 | 14 | 111 |
| CDR-H2 | FIRNKANSGTTEYAASVKG | 50-68 | 19 | 112 |
| HFR3 | RFTISRDDSKNTLYLQMNSLRAEDTAVYYCAR | 69-100 | 32 | 113 |
| CDR-H3 | DRGLRFYFDY | 101-110 | 10 | 114 |
| HFR4 | WGQGTTVTVSS | 111-121 | 11 | 115 |

### TLR Agonist Adjuvant Compounds

The immunoconjugate of the invention comprises an immunostimulatory, TLR agonist adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-KB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I—KB. As a result, NF-κB enters the cell nucleus and initiates transcription of genes whose promoters contain NF-κB binding sites, such as cytokines. Additional modes of regulation for TLR signaling include TTR-domain containing adapter-inducing interferon-0 (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-7, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

TLR agonist adjuvant moieties include, but are not limited to, compounds: (a) imidazo[4,5-c]quinolin-4-amine (WO 2020/190762, WO 2020/190725, WO 2019/222676, WO 2018/112108, WO 2018/009916); (b) quinolin-2-amine (WO 2021/046112); (c) 2-amino-3H-benzo[b]azepine-4-carboxamide (WO 2020/252254, WO 2020/252294); (d) 5-amino-6H-thieno[3,2-b]azepine-7-carboxamide (WO 2021/081407, WO 2021/081402); (e) 5-amino-1,6-dihydropyrazolo[4,3-b]azepine-7-carboxamide; and (f) 5-amino-2,6-dihydropyrazolo[4,3-b]azepine-7-carboxamide, having formulas a-f:

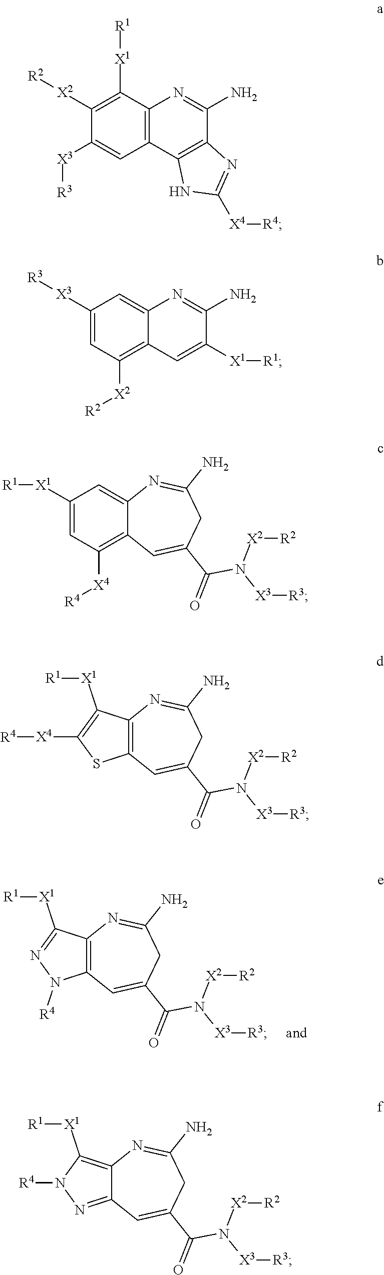

with R¹⁻⁴ and X¹⁻⁴ substituents as described herein.

### TLR Agonist-Linker Compounds

The immunoconjugates of the invention are prepared by conjugation of a cysteine-mutant antibody with a TLR agonist-linker (TLR-L) compound. The TLR-L compounds comprise a TLR agonist moiety covalently attached to a linker unit. The linker unit comprises functional groups and subunits which affect stability, permeability, solubility, and other pharmacokinetic, safety, and efficacy properties of the immunoconjugates. The linker unit includes a reactive electrophilic functional group such as maleimide or bromoacetamide which reacts, i.e. conjugates, with a reactive cysteine thiol group of the cysteine-mutant antibody to form the immunoconjugate.

Where the reactive electrophilic functional group is maleimide, the resulting succinimide ring in the linker is susceptible to ring-opening reactions via hydrolysis, especially at high pH and elevated temperatures. Once the succinimide ring is opened, in vivo stability and the therapeutic activity may be modulated (Zheng, K. et al (2019) J Pharm Sci, 108(1):133-141).

Electrophilic reactive functional groups suitable for the TLR-L compounds include, but are not limited to, maleimides (thiol reactive); halogenated acetamides such as iodoacetamide, bromoacetamide, and chloroacetamide (thiol reactive); vinyl sulfones (thiol, amine, and hydroxyl reactive); and pyridyl disulfides (thiol reactive). Further reagents include, but are not limited to, those described in Hermanson, Bioconjugate Techniques 2^{nd} Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Considerations for the design of the immunoconjugates of the invention include: (1) preventing the premature release of the TLR agonist moiety during in vivo circulation and (2) ensuring that a biologically active form of the TLR agonist moiety is released at the desired site of action at an adequate rate. The complex structure of the immunoconjugate together with its functional properties requires careful design and selection of every component of the molecule including antibody, conjugation site, linker structure, and the pyrazoloazepine compound. The linker determines the mechanism and rate of adjuvant release.

Generally, the linker unit (L) may be cleavable or non-cleavable. Cleavable linker units may include a peptide sequence which is a substrate for certain proteases such as Cathepsins which recognize and cleave the peptide linker unit, separating the TLR agonist from the antibody (Caculitan N G, et al (2017) Cancer Res. 77(24):7027-7037).

Cleavable linker units may include labile functionality such as an acid-sensitive disulfide group (Kellogg, B A et al (2011) Bioconjugate Chem. 22, 717-727; Ricart, A. D. et al (2011) Clin. Cancer Res. 17, 6417-6427; Pillow, T., et al (2017) Chem. Sci. 8, 366-370; Zhang D, et al (2016) ACS Med Chem Lett. 7(11):988-993).

In some embodiments, the linker is non-cleavable under physiological conditions. As used herein, the term “physiological conditions” refers to a temperature range of 20-40 degrees Celsius, atmospheric pressure (i.e. 1 atm), a pH of about 6 to about 8, and the one or more physiological enzymes, proteases, acids, and bases. One advantage of a non-cleavable linker between the antibody and TLR agonist moiety in an immunoconjugate is minimizing premature payload release and corresponding toxicity.

In one embodiment, the invention includes a peptide linking unit, PEP, between the cell-binding agent and the immunostimulatory TLR agonist moiety, comprising a peptide radical based on a linear sequence of specific amino acid residues which can be selectively cleaved by a protease such as a cathepsin, a tumor-associated elastase enzyme or an enzyme with protease-like or elastase-like activity. The peptide radical may be about two to about twelve amino acids.

Enzymatic cleavage of a bond within the peptide linker releases an active form of the immunostimulatory TLR agonist moiety. This leads to an increase in the tissue specificity of the conjugates according to the invention and thus to an additional decrease of toxicity of the conjugates according to the invention in other tissue types.

In an exemplary embodiment, PEP is comprised of amino acid residues (AA) of amino acids selected from the group consisting of:

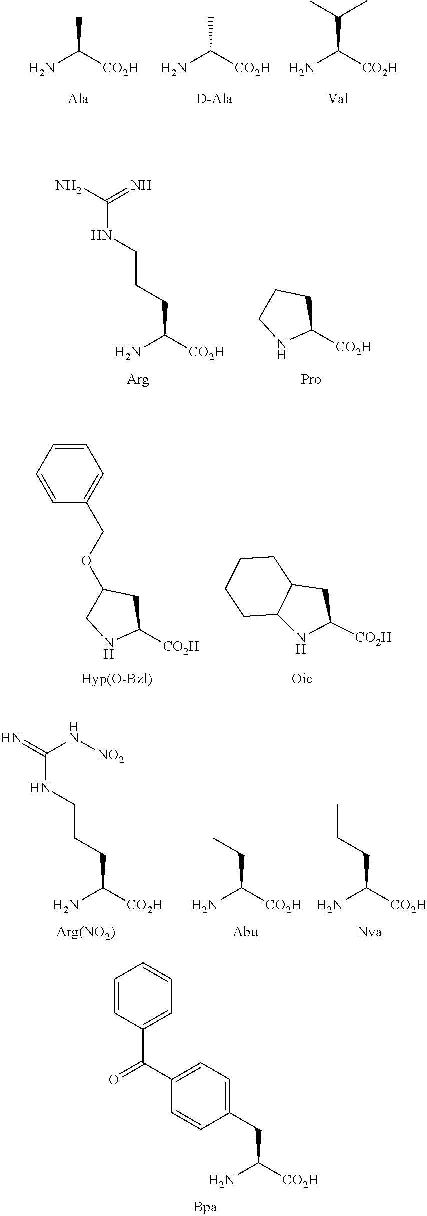

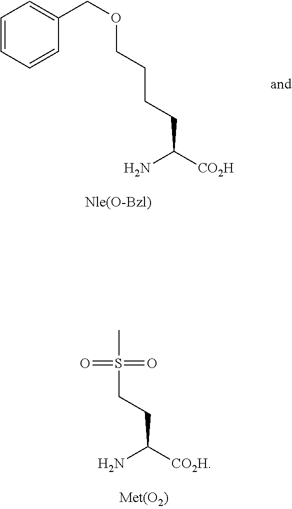 [SMILES: N[C@@H](CCCCOCc1ccccc1)C(=O)O ; CS(=O)(=O)CC[C@H](N)C(=O)O]

In an exemplary embodiment, PEP is selected from the group consisting of Ala-Pro-Val, Asn-Pro-Val, Ala-Ala-Val, Ala-Ala-Pro-Ala, Ala-Ala-Pro-Val, and Ala-Ala-Pro-Nva.

In an exemplary embodiment, PEP has the formula:

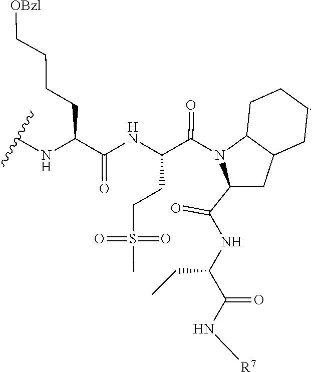

In an exemplary embodiment, PEP has the formula:

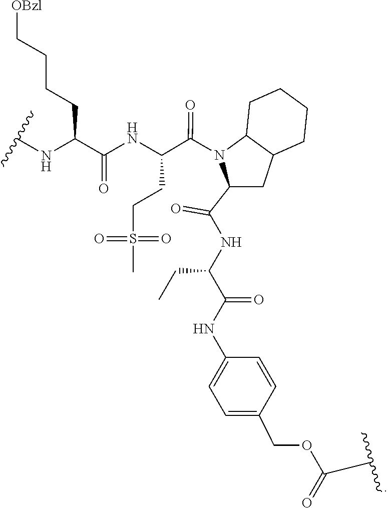

In an exemplary embodiment, PEP is selected from the formulas:

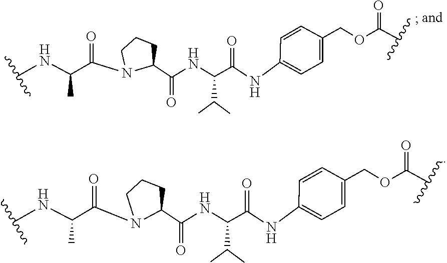 [SMILES: *N[C@H](C)C(=O)N1CCC[C@H]1C(=O)N[C@H](C(=O)Nc1ccc(COC(*)=O)cc1)C(C)C ; *N[C@@H](C)C(=O)N1CCC[C@H]1C(=O)N[C@H](C(=O)Nc1ccc(COC(*)=O)cc1)C(C)C]

The linker provides sufficient stability of the immunoconjugate in biological media, e.g. culture medium or serum and, at the same time, the desired intracellular action within tumor tissue as a result of its specific enzymatic or hydrolytic cleavability with release of the immunostimulatory TLR agonist moiety, i.e. “payload”.

The enzymatic activity of a protease, cathepsin, or elastase can catalyze cleavage of a covalent bond of the immunoconjugate under physiological conditions. The enzymatic activity being the expression product of cells associated with tumor tissue. The enzymatic activity on the cleavage site of the targeting peptide converts the immunoconjugate to an active immunostimulatory drug free of targeting peptide and linking group. The cleavage site may be specifically recognized by the enzyme. Cathepsin or elastase may catalyze the cleavage of a specific peptidyl bond between the C-terminal amino acid residue of the specific peptide and the immunostimulatory TLR agonist moiety of the immunoconjugate.

In one embodiment, the TLR agonist-linker (TLR-L) compound includes a linking unit, i.e. L or linker, between the cell-binding agent and the TLR agonist moiety, comprising a substrate for glucuronidase (Jeffrey S C, et al (2006) Bioconjug. Chen. 17(3):-831-40), or sulfatase (Bargh J D, et al (2020) Chem Sci. I1(9):2375-2380) cleavage. In particular, L include a Gluc unit and comprise a formula selected from:

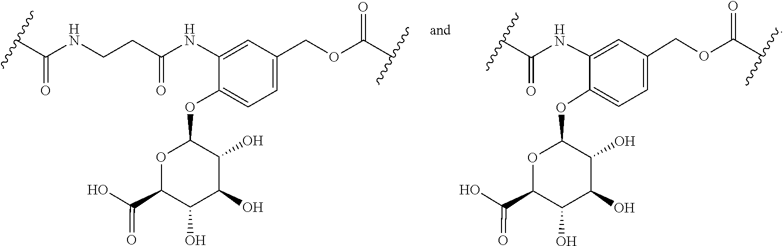 [SMILES: *C(=O)NCCC(=O)Nc1cc(COC(*)=O)ccc1O[C@@H]1O[C@H](C(=O)O)[C@@H](O)[C@H](O)[C@H]1O ; *C(=O)Nc1cc(COC(*)=O)ccc1O[C@@H]1O[C@H](C(=O)O)[C@@H](O)[C@H](O)[C@H]1O]

Specific cleavage of the immunoconjugates of the invention takes advantage of the presence of tumor infiltrating cells of the immune system and leukocyte-secreted enzymes, to promote the activation of an anticancer drug at the tumor site.

Exemplary embodiments of a TLR agonist-linker (TLR-L) compound is selected from formulas a-f:

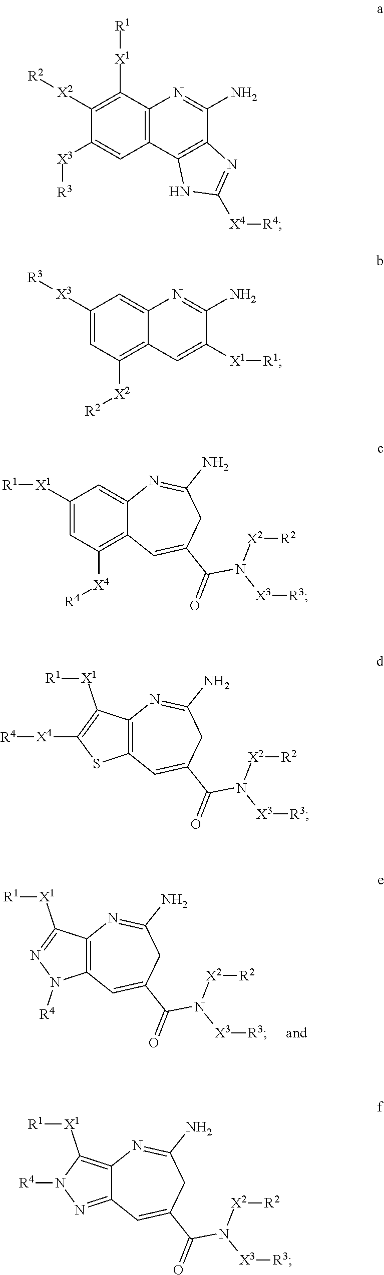

wherein X¹, X², X³ and X⁴ are independently selected from the group consisting of a bond, C(═O), C(═O)N(R⁵), O, N(R⁵), S, S(O)₂, and S(O)₂N(R⁵);
R¹, R², R³, and R⁴ are independently selected from the group consisting of H, C₁-C₁₂ alkyl, C₂-C₆ alkenyl, C₂-C₆ alkynyl, C3-C12 carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-N(R⁵)₂;
—(C₁-C₂₀ heteroaryldiyl)-NR⁵—C(═NR^{5a})N(R⁵)—*;
—(C₁-C₂₀ heteroaryldiyl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-*;
—C(═O)N(R⁵)—(C₁-C₁₂ alkyldiyl)-C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—N(R⁵)CO₂(R⁵)—*;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—OC(═O)N(R⁵)₂;
—OC(═O)N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and —S(═O)₂—(C₂-C₂₀ heterocyclyldiyl)-(C₁-C₁₂ alkyldiyl)-OH;
or R² and R³ together form a 5- or 6-membered heterocyclyl ring;
R⁵ is selected from the group consisting of H, C₆-C₂₀ aryl, C₃-C₁₂ carbocyclyl, C₂-C₂₀ heterocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
   Q-C(═O)—PEG-;
   Q-C(═O)—PEG-C(═O)N(R⁶)—(C₁-C₁₂ alkyldiyl)-C(═O)-Gluc-;
   Q-C(═O)—PEG-O—;
   Q-C(═O)—PEG-O—C(═O)—;
   Q-C(═O)—PEG-C(═O)—;
   Q-C(═O)—PEG-C(═O)—PEP-;
   Q-C(═O)—PEG-N(R⁶)—;
   Q-C(═O)—PEG-N(R⁶)—C(═O)—;
   Q-C(═O)—PEG-N(R⁶)—PEG-C(═O)—PEP-;
   Q-C(═O)—PEG-N+(R⁶)₂-PEG-C(═O)—PEP-;
   Q-C(═O)—PEG-C(═O)—PEP-N(R⁶)—(C₁-C₁₂ alkyldiyl)-;
   Q-C(═O)—PEG-C(═O)—PEP-N(R⁶)—(C₁-C₁₂ alkyldiyl)N(R⁶)C(═O)—(C₂-C₅ monoheterocyclyldiyl)-;
   Q-C(═O)—PEG-SS—(C₁-C₁₂ alkyldiyl)-OC(═O)—;
   Q-C(═O)—PEG-SS—(C₁-C₁₂ alkyldiyl)-C(═O)—;
   Q-C(═O)—(C₁-C₁₂ alkyldiyl)-C(═O)—PEP-;
   Q-C(═O)—(C₁-C₁₂ alkyldiyl)-C(═O)—PEP-N(R⁶)—(C₁-C₁₂ alkyldiyl)-;
   Q-C(═O)—(C₁-C₁₂ alkyldiyl)-C(═O)—PEP-N(R⁶)—(C₁-C₁₂ alkyldiyl)-N(R⁵)—C(═O);
   Q-C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
   Q-(CH₂)ₘC(═O)N(R⁶)—PEG-;
   Q-(CH₂)ₘC(═O)N(R⁶)—PEG-C(═O)N(R⁶)—(C₁-C₁₂ alkyldiyl)-C(═O)-Gluc-;
   Q-(CH₂)ₘC(═O)N(R⁶)—PEG-O—;
   Q-(CH₂)ₘC(═O)N(R⁶)—PEG-O—C(═O)—;
   Q-(CH₂)ₘC(═O)N(R⁶)—PEG-C(═O)—;
   Q-(CH₂)ₘC(═O)N(R⁶)—PEG-N(R⁵)—;
   Q-(CH₂)ₘC(═O)N(R⁶)—PEG-N(R⁵)—C(═O)—;
   Q-(CH₂)ₘC(═O)N(R⁶)—PEG-C(═O)—PEP-;
   Q-(CH₂)ₘC(═O)N(R⁶)—PEG-SS—(C₁-C₁₂ alkyldiyl)-OC(═O)—;
   Q-(CH₂)ₘC(═O)—PEP-N(R⁶)—(C₁-C₁₂ alkyldiyl)-;
   Q-(CH₂)ₘC(═O)—PEP-N(R⁶)—(C₁-C₁₂ alkyldiyl)N(R⁶)C(═O)—; and
   Q-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;

Gluc has the formula:

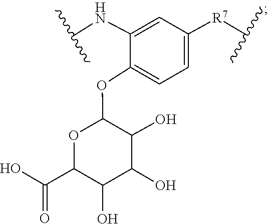

PEP has the formula:

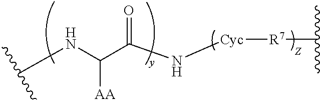

where AA is independently selected from a natural or unnatural amino acid side chain, or one or more of AA, and an adjacent nitrogen atom form a 5-membered ring proline amino acid, and the wavy line indicates a point of attachment;

Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

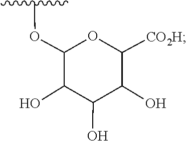 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
Q is selected from the group consisting of maleimide, bromoacetamide, and pyridyldisulfide;
where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C═CH, —C═CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

Exemplary embodiments of a TLR agonist-linker (TLR-L) compound include wherein Q is maleimide.

An exemplary embodiment of a TLR agonist-linker (TLR-L) compound is selected from Table 8. Each TLR-L compound was characterized by mass spectrometry and shown to have the mass indicated. The TLR-L compounds of Tables 8 and 9 demonstrate the surprising and unexpected property of TLR8 agonist selectivity which may predict useful therapeutic activity to treat cancer and other disorders.

Comparator compounds from Table 9 have an activated ester, tetrafluorophenyl or sulfotetrafluorophenyl group which reacts with a lysine residue of an antibody to form an immunoconjugate with an amide bond between the antibody and the TLR-agonist-linker moiety according to Example 203.

TABLE 8
TLR agonist-linker (TLR-L) compounds

| TLR-L | | |
|---|---|---|
| No. | Structure | MW |
| TLR-L- 1 | 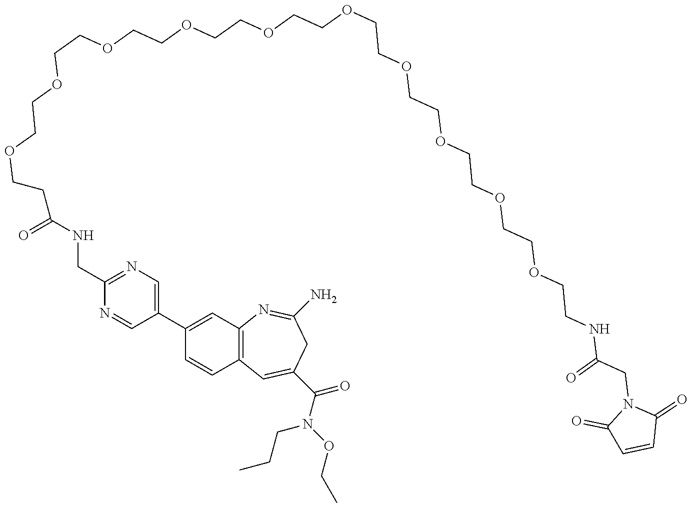 [SMILES: CCCN(OCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1043.2 |
| TLR-L- 2 | 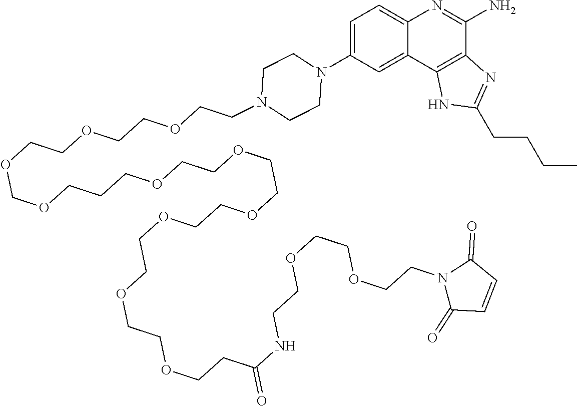 [SMILES: CCCCc1nc2c(N)nc3ccc(N4CCN(CCOCCOCCOCOCCCOCCOCCOCCOCCOCCOCCC(=O)NCCOCCOCCN5C(=O)C=CC5=O)CC4)cc3c2[nH]1] | 1047.3 |
| TLR-L- 3 | 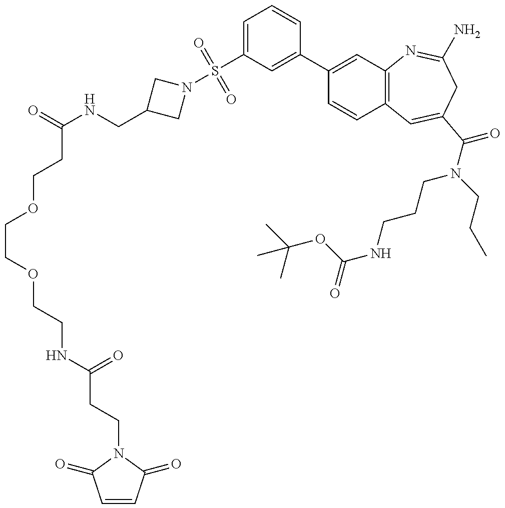 [SMILES: CCCN(CCCNC(=O)OC(C)(C)C)C(=O)C1=Cc2ccc(-c3cccc(S(=O)(=O)N4CC(CNC(=O)CCOCCOCCNC(=O)CCN5C(=O)C=CC5=O)C4)c3)cc2N=C(N)C1] | 935.1 |
| TLR-L- 4 | 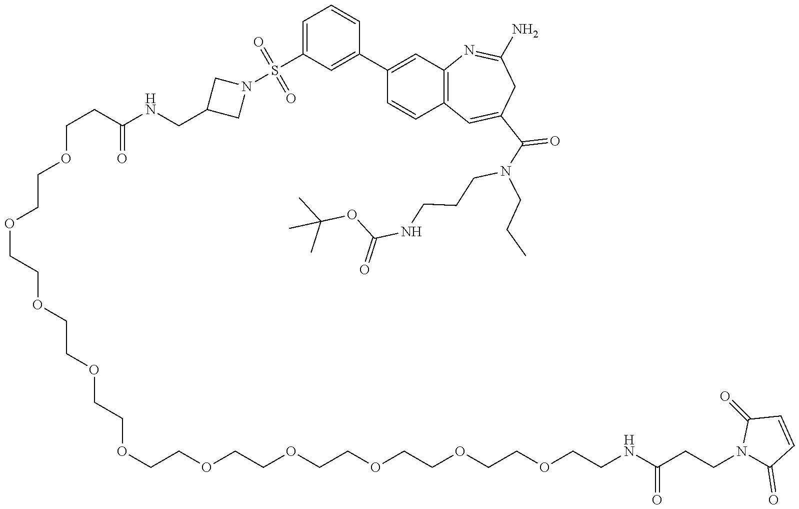 [SMILES: CCCN(CCCNC(=O)OC(C)(C)C)C(=O)C1=Cc2ccc(-c3cccc(S(=O)(=O)N4CC(CNC(=O)CCOCCOCCOCCOCCOCCOCCOCCOCCOCCOCCNC(=O)CCN5C(=O)C=CC5=O)C4)c3)cc2N=C(N)C1] | 1287.5 |
| TLR-L- 5 | 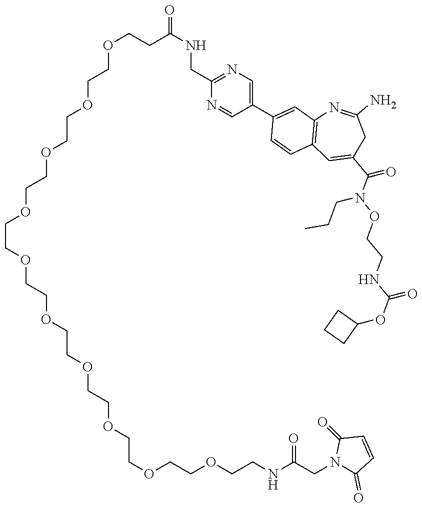 [SMILES: CCCN(OCCNC(=O)OC1CCC1)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1] | 1156.3 |
| TLR-L- 6 | 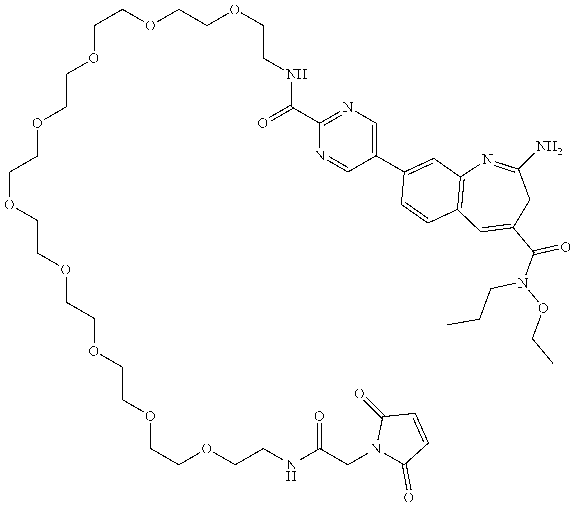 [SMILES: CCCN(OCC)C(=O)C1=Cc2ccc(-c3cnc(C(=O)NCCOCCOCCOCCOCCOCCOCCOCCOCCOCCNC(=O)CN4C(=O)C=CC4=O)nc3)cc2N=C(N)C1] | 985.1 |
| TLR-L- 7 | 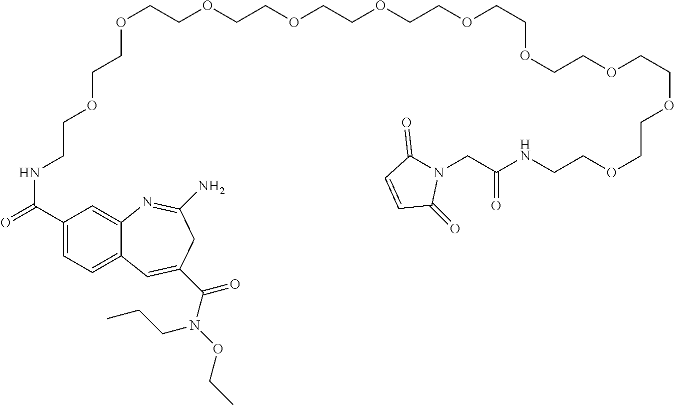 [SMILES: CCCN(OCC)C(=O)C1=Cc2ccc(C(=O)NCCOCCOCCOCCOCCOCCOCCOCCOCCOCCOCCNC(=O)CN3C(=O)C=CC3=O)cc2N=C(N)C1] | 951.1 |

TABLE 9
TLR agonist-linker comparator compounds

| Comp | Structure | MW |
|---|---|---|
| C-1 | 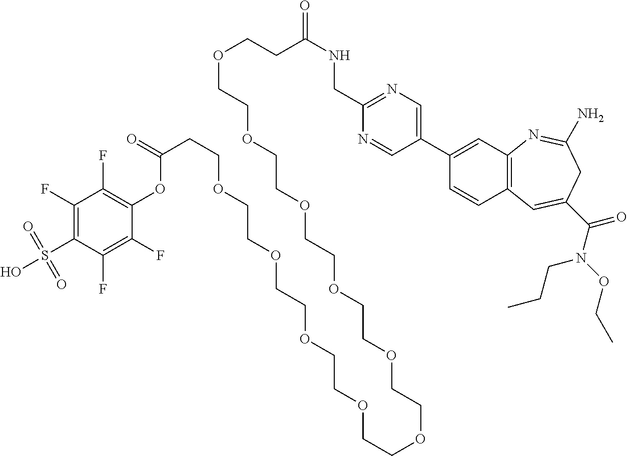 [SMILES: CCCN(OCC)C(=O)C1=Cc2ccc(-c3cnc(CNC(=O)CCOCCOCCOCCOCCOCCOCCOCCOCCOCCOCCC(=O)Oc4c(F)c(F)c(S(=O)(=O)O)c(F)c4F)nc3)cc2N=C(N)C1] | 1163.2 |
| C-2 | 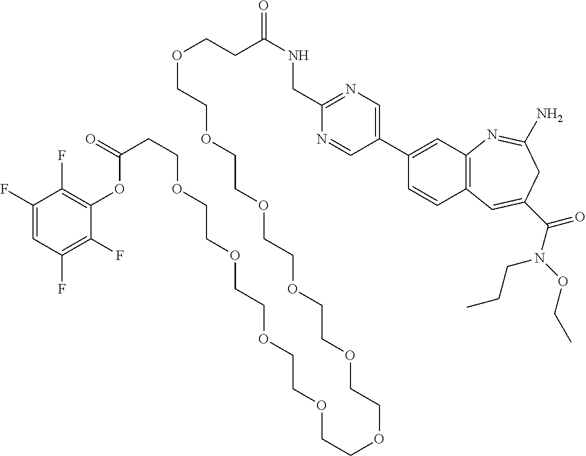 [SMILES: CCCN(OCC)C(=O)C1=Cc2ccc(-c3cnc(CNC(=O)CCOCCOCCOCCOCCOCCOCCOCCOCCOCCOCCC(=O)Oc4c(F)c(F)cc(F)c4F)nc3)cc2N=C(N)C1] | 1083.1 |

### Immunoconjugates

The immunoconjugates of the invention comprise a cysteine-mutant antibody covalently attached to one or more TLR agonist moieties by a linker.

Exemplary embodiments of immunoconjugates comprise a cysteine-mutant antibody with a cysteine mutation in the hinge region.

Exemplary embodiments of immunoconjugates comprise a cysteine-mutant antibody with a cysteine mutation selected from the group consisting of: K145C, S1114C, E105C, S157C, L174C, G178C, S159C, V191C, L201C, S119C, V167C, I199C, T129C, Q196C, A378C, K149C, K188C, and A140C, numbered according to the EU format.

Exemplary embodiments of immunoconjugates have Formula I:

Ab-[L-D]ₚ I
or a pharmaceutically acceptable salt thereof,
wherein:
Ab is the cysteine-mutant antibody;
p is an integer from 1 to 8;
L is the linker; D is the TLR agonist moiety selected from formulas a-f:

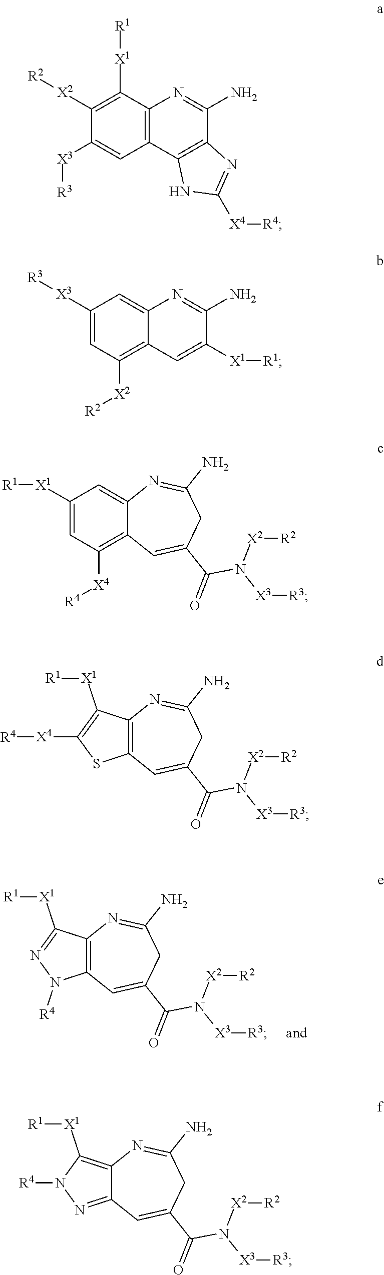

X¹, X², X³ and X⁴ are independently selected from the group consisting of a bond, C(═O), C(═O)N(R⁵), O, N(R⁵), S, S(O)₂, and S(O)₂N(R⁵);
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-N(R⁵)₂;
—(C₁-C₂₀ heteroaryldiyl)-NR⁵—C(═NR^{5a})N(R⁵)—*;
—(C₁-C₂₀ heteroaryldiyl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-*;
—C(═O)N(R⁵)—(C₁-C₁₂ alkyldiyl)-C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—N(R⁵)CO₂(R⁵)—*;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₁₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—OC(═O)N(R⁵)₂;
—OC(═O)N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
R⁵ is selected from the group consisting of H, C₆-C₂₀ aryl, C₃-C₁₂ carbocyclyl, C₂-C₂₀ heterocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
   —C(═O)—PEG-;
   —C(═O)—PEG-C(═O)N(R⁶)—(C₁-C₁₂ alkyldiyl)-C(═O)-Gluc-;
   —C(═O)—PEG-O—;
   —C(═O)—PEG-O—C(═O)—;
   —C(═O)—PEG-C(═O)—;
   —C(═O)—PEG-C(═O)—PEP-;
   —C(═O)—PEG-N(R⁶)—;
   —C(═O)—PEG-N(R⁶)—C(═O)—;
   —C(═O)—PEG-N(R⁶)—PEG-C(═O)—PEP-;
   —C(═O)—PEG-N+(R⁶)₂-PEG-C(═O)—PEP-;
   —C(═O)—PEG-C(═O)—PEP-N(R⁶)—(C₁-C₁₂ alkyldiyl)-;
   —C(═O)—PEG-C(═O)—PEP-N(R⁶)—(C₁-C₁₂ alkyldiyl)N(R⁶)C(═O)—(C₂-C₅ monoheterocyclyldiyl)-;
   —C(═O)—PEG-SS—(C₁-C₁₂ alkyldiyl)-OC(═O)—;
   —C(═O)—PEG-SS—(C₁-C₁₂ alkyldiyl)-C(═O)—;
   —C(═O)—(C₁-C₁₂ alkyldiyl)-C(═O)—PEP-;
   —C(═O)—(C₁-C₁₂ alkyldiyl)-C(═O)—PEP-N(R⁶)—(C₁-C₁₂ alkyldiyl)-;
   —C(═O)—(C₁-C₁₂ alkyldiyl)-C(═O)—PEP-N(R⁶)—(C₁-C₁₂ alkyldiyl)-N(R⁵)—C(═O);
   —C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
   -succinimidyl-(CH₂)ₘ—C(═O)N(R⁶)—PEG-;
   -succinimidyl-(CH₂)ₘ—C(═O)N(R⁶)—PEG-C(═O)N(R⁶)—(C₁-C₁₂
   alkyldiyl)-C(═O)-Gluc-;
   -succinimidyl-(CH₂)ₘ—C(═O)N(R⁶)—PEG-O—;
   -succinimidyl-(CH₂)ₘ—C(═O)N(R⁶)—PEG-O—C(═O)—;
   -succinimidyl-(CH₂)ₘ—C(═O)N(R⁶)—PEG-C(═O)—;
   -succinimidyl-(CH₂)ₘ—C(═O)N(R⁶)—PEG-N(R⁵)—;
   -succinimidyl-(CH₂)ₘ—C(═O)N(R⁶)—PEG-N(R⁵)—C(═O)—;
   -succinimidyl-(CH₂)ₘ—C(═O)N(R⁶)—PEG-C(═O)—PEP-;
   -succinimidyl-(CH₂)ₘ—C(═O)N(R⁶)—PEG-SS—(C₁-C₁₂ alkyldiyl)-OC(═O)—;
   -succinimidyl-(CH₂)ₘ—C(═O)—PEP-N(R⁶)—(C₁-C₁₂ alkyldiyl)-;
   -succinimidyl-(CH₂)ₘ—C(═O)—PEP-N(R⁶)—(C₁-C₁₂ alkyldiyl)N(R⁶)C(═O)—; and
   -succinimidyl-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;

Gluc has the formula:

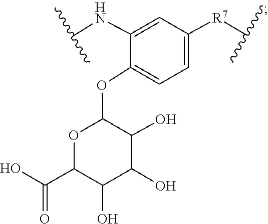

PEP has the formula:

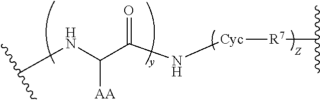

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

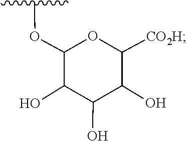 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C—CH, —C—CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

Exemplary embodiments of immunoconjugates include wherein X¹ is a bond, and R¹ is H.

Exemplary embodiments of immunoconjugates include wherein X² is a bond, and R² is C₁-C₈ alkyl.

Exemplary embodiments of immunoconjugates include wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₅ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

Exemplary embodiments of immunoconjugates include wherein R² is C₁-C₈ alkyl and R³ is —(C₁-C₅ alkyldiyl)-N(R⁵)CO₂R4.

Exemplary embodiments of immunoconjugates include wherein R² is —CH₂CH₂CH₃ and R³ is selected from —CH₂CH₂CH₂NHCO₂(t-Bu), —OCH₂CH₂NHCO₂(cyclobutyl), and —CH₂CH₂CH₂NHCO₂(cyclobutyl).

Exemplary embodiments of immunoconjugates include wherein R² and R³ are each independently selected from —CH₂CH₂CH₃, —OCH₂CH₃, —OCH₂CF₃, —CH₂CH₂CF₃, —OCH₂CH₂OH, and —CH₂CH₂CH₂OH.

Exemplary embodiments of immunoconjugates include wherein R² and R³ are each —CH₂CH₂CH₃.

Exemplary embodiments of immunoconjugates include wherein R² is —CH₂CH₂CH₃ and R³ is —OCH₂CH₃.

Exemplary embodiments of immunoconjugates include wherein X³—R³ is selected from the group consisting of:

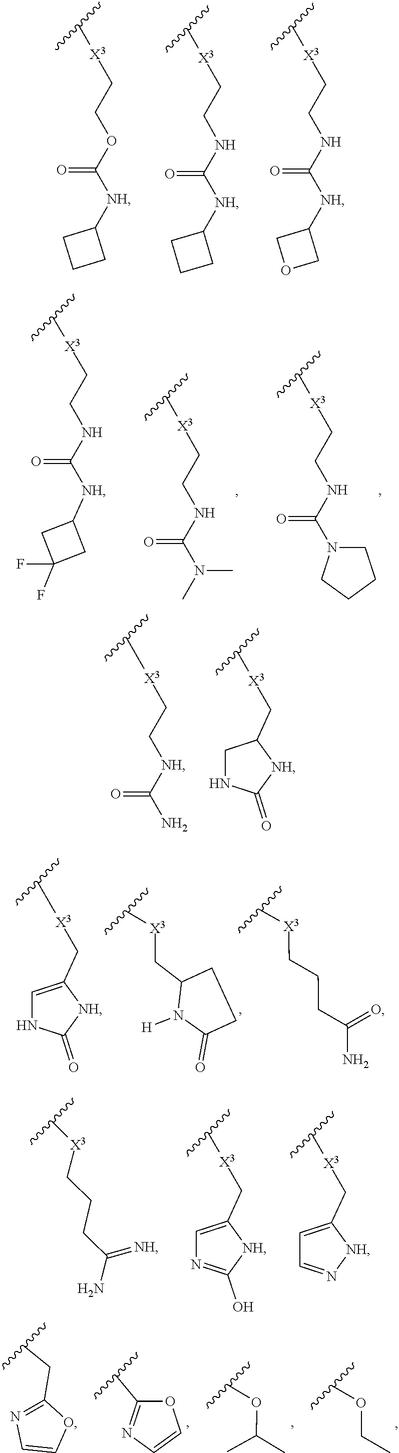

Exemplary embodiments of immunoconjugates include where R² or R³ is attached to L.

Exemplary embodiments of immunoconjugates include wherein X³—R³-L is selected from the group consisting of:

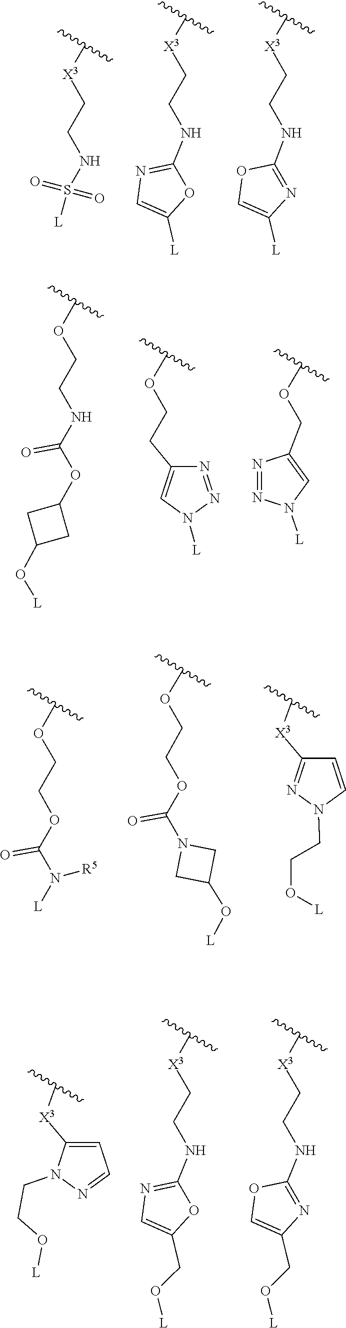

where the wavy line indicates the point of attachment to N.

Exemplary embodiments of immunoconjugates include wherein R⁴ is C₁-C₁₂ alkyl.

Exemplary embodiments of immunoconjugates include wherein R⁴ is —(C₁-C₁₂ alkyldiyl)-N(R⁵)—*; where the asterisk * indicates the attachment site of L.

Exemplary embodiments of immunoconjugates include wherein L is —C(═O)—PEG- or —C(═O)—PEG-C(═O)—.

Exemplary embodiments of immunoconjugates include wherein L is attached to a cysteine thiol of the antibody.

Exemplary embodiments of immunoconjugates include wherein for the PEG, m is 1 or 2, and n is an integer from 2 to 10.

Exemplary embodiments of immunoconjugates include wherein n is 10.

Exemplary embodiments of immunoconjugates include wherein L comprises PEP and PEP is a dipeptide and has the formula:

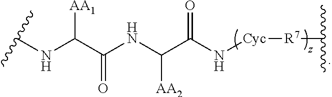

Exemplary embodiments of immunoconjugates include wherein PEP has the formula:

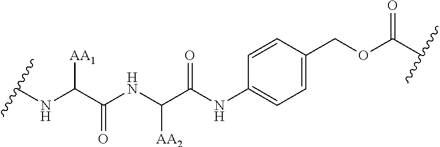

where AA₁ and AA₂ are independently selected from a side chain of a naturally-occurring amino acid.

Exemplary embodiments of immunoconjugates include wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂; or AA₁ and AA₂ form a 5-membered ring proline amino acid.

Exemplary embodiments of immunoconjugates include wherein AA₁ is —CH(CH₃)₂, and AA₂ is —CH₂CH₂CH₂NHC(O)NH₂.

Exemplary embodiments of immunoconjugates include wherein AA₁ and AA₂ are independently selected from GlcNAc aspartic acid, —CH₂SO₃H, and —CH₂OPO₃H.

Exemplary embodiments of immunoconjugates include wherein L comprises PEP and PEP is a tripeptide and has the formula:

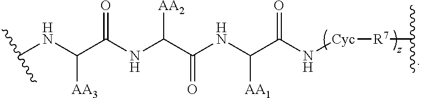

Exemplary embodiments of immunoconjugates include wherein L comprises PEP and PEP is a tetrapeptide and has the formula:

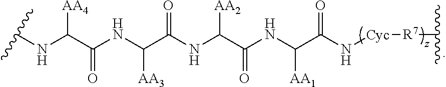

Exemplary embodiments of immunoconjugates include wherein the PEP tetrapeptide is selected from:
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

Exemplary embodiments of immunoconjugates include wherein L comprises PEP and PEP is selected from the group consisting of Ala-Pro-Val, Asn-Pro-Val, Ala-Ala-Val, Ala-Ala-Pro-Ala, Ala-Ala-Pro-Val, and Ala-Ala-Pro-Nva.

Exemplary embodiments of immunoconjugates include wherein L comprises PEP and PEP is selected from the structures:

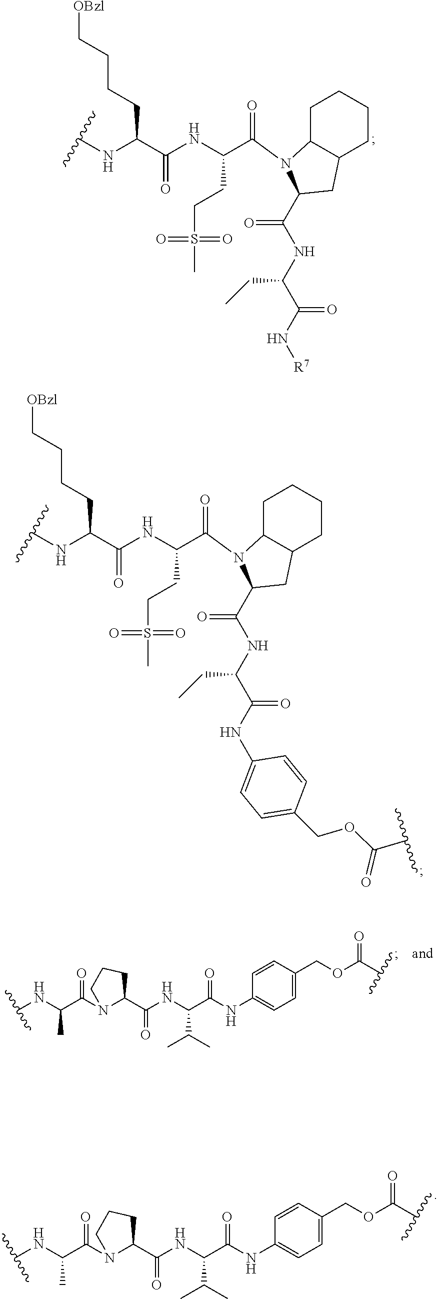

Exemplary embodiments of immunoconjugates include wherein L is selected from the structures:

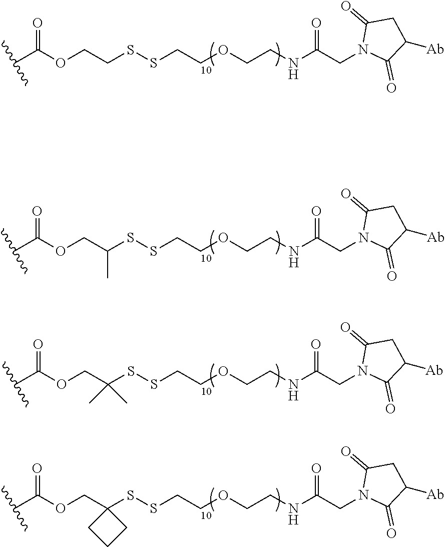

where the wavy line indicates the attachment to R⁵.

Exemplary embodiments of immunoconjugates of the invention of Table 10 were prepared by conjugation of a cysteine-mutant antibody with a TLR agonist-linker compound from Table 8.

The comparator amide-linked immunoconjugate Lys IC-1 from Table 11 was prepared by conjugation of trastuzumab with TLR agonist-linker comparator compound C-1 from Table 9.

Each immunoconjugate of Tables 10a, 10b and 11 was prepared according to the methods of Examples 202 and 203 respectively, purified by HPLC, and characterized by mass spectroscopy.

TABLE 10a
Cysteine-mutant TLR Immunoconjugates (IC)

| | | | | | CDC |
|---|---|---|---|---|---|
| | | | | | Activation |
| Immuno- | | | Cys | | (IL12p70 |
| conjugate | TLR-L | Antibody | Mutant | | Secretion)- |
| No. | Table 8 | Target | site | DAR | EC₅₀ (nM) |
| IC-1 | TLR-L-1 | Trastuzumab | K145C | 2.0 | 1.8 |
| | | HER2 | | | |
| IC-2 | TLR-L-1 | Trastuzumab | S114C | 2.0 | 6.2 |
| | | HER2 | | | |
| IC-3 | TLR-L-1 | Trastuzumab | E105C | 2.0 | N/A |
| | | HER2 | | | |
| IC-4 | TLR-L-1 | Trastuzumab | S157C | 2.0 | 5.1 |
| | | HER2 | | | |
| IC-5 | TLR-L-1 | Trastuzumab | L174C | 2.0 | 7.4 |
| | | HER2 | | | |
| IC-6 | TLR-L-1 | Trastuzumab | G178C | 2.0 | 7.2 |
| | | HER2 | | | |
| IC-7 | TLR-L-1 | Trastuzumab | S159C | 1.8 | 2.8 |
| | | HER2 | | | |
| IC-8 | TLR-L-1 | Trastuzumab | V191C | 2.0 | 1.1 |
| | | HER2 | | | |
| IC-9 | TLR-L-1 | Trastuzumab | L201C | 2.0 | 3.2 |
| | | HER2 | | | |
| IC-10 | TLR-L-1 | Trastuzumab | S119C | 1.8 | 1.5 |
| | | HER2 | | | |
| IC-11 | TLR-L-1 | Trastuzumab | V167C | 1.9 | N/A |
| | | HER2 | | | |
| IC-12 | TLR-L-1 | Trastuzumab | I199C | 2.0 | 3.3 |
| | | HER2 | | | |
| IC-13 | TLR-L-1 | Trastuzumab | T129C | 1.7 | 0.6 |
| | | HER2 | | | |
| IC-14 | TLR-L-1 | Trastuzumab | Q196C | 1.8 | 1.6 |
| | | HER2 | | | |
| IC-15 | TLR-L-1 | Trastuzumab | A378C | 1.9 | 9.4 |
| | | HER2 | | | |
| IC-16 | TLR-L-1 | Trastuzumab | K149C | 2.0 | 1.1 |
| | | HER2 | | | |
| IC-17 | TLR-L-1 | Trastuzumab | K188C | 2.0 | 0.6 |
| | | HER2 | | | |
| IC-18 | TLR-L-1 | Trastuzumab | A140C | 1.9 | 1.7 |
| | | HER2 | | | |

TABLE 10b
Cysteine-mutant TLR Immunoconjugates (IC)

| | | | | | CDC |
|---|---|---|---|---|---|
| | | | | | Activation |
| | | | | | (IL12p70 |
| Immuno- | TLR-L | | Cys | | Secretion)- |
| conjugate | Tables 8, | Antibody | Mutant | | EC₅₀ |
| No. | 9 | Target | site | DAR | (nM) |
| IC-19 | TLR-L-2 | Trastuzumab | V205C | 1.9 | |
| | | HER2 | | | |
| IC-20 | TLR-L-3 | Trastuzumab | K107C | 2.0 | |
| | | HER2 | | | |
| IC-21 | TLR-L-4 | Trastuzumab | K107C | 2.0 | |
| | | HER2 | | | |
| IC-22 | TLR-L-3 | Trastuzumab | K414C | 2.0 | |
| | | HER2 | | | |
| IC-23 | TLR-L-3 | Trastuzumab | V205C | 1.9 | |
| | | HER2 | | | |
| IC-24 | TLR-L-4 | Trastuzumab | V205C | 1.9 | |
| | | HER2 | | | |
| IC-25 | TLR-L-5 | Trastuzumab | K107C | 2.0 | |
| | | HER2 | | | |
| IC-26 | TLR-L-5 | Trastuzumab | K414C | 2.0 | |
| | | HER2 | | | |
| IC-27 | TLR-L-1 | TROP2 | V191C | 1.9 | 2.7 |
| IC-28 | TLR-L-1 | TROP2 | T129C | 1.8 | 2336 |
| IC-29 | TLR-L-1 | TROP2 | K188C | 1.8 | 2.3 |
| IC-30 | TLR-L-1 | PD-L1 | S119C | 1.7 | 0.3 |
| IC-31 | TLR-L-1 | PD-L1 | K188C | 2.0 | 0.2 |
| IC-32 | TLR-L-1 | TROP2 | S119C | 2.0 | 139 |
| IC-33 | TLR-L-6 | PD-L1 | K188C | 1.2 | 0.8 |
| IC-34 | TLR-L-7 | PD-L1 | K188C | 2.0 | |
| IC-35 | TLR-L-7 | Trastuzumab | K188C | 1.8 | |
| | | HER2 | | | |

TABLE 11
Comparator Immunoconjugates (IC)

| Lysine | | | | cDC |
|---|---|---|---|---|
| conjugated | | | | Activation |
| Comparator | | | | (IL12p70 |
| Immuno- | | | | Secretion)- |
| conjugate | Comparator | | | EC₅₀ |
| No. | Table 8 | Antibody | DAR | (nM) |
| Lys IC-1 | C-1 | trastuzumab | 2.4 | 1.7 |
| Lys IC-2 | C-1 | TROP2 | 3.2, 2.4 | |
| Lys IC-3 | C-1 | PD-L1 | 2.4 | 0.3 |

### Biological Activity of Immunoconjugates

Immunoconjugates from Tables 10a, 10b and 11 were tested for activity by the assays of Example 204. Dendritic Cell Based assays are useful for the evaluation of cancer immunotherapies. Dendritic cells (DC) are specialized antigen-presenting cells bridging the innate and adaptive immune systems and which mediate immunity and tolerance. Immunoconjugates were assayed by the conventional/classical dendritic cell (cDC) assay described in Example 204.

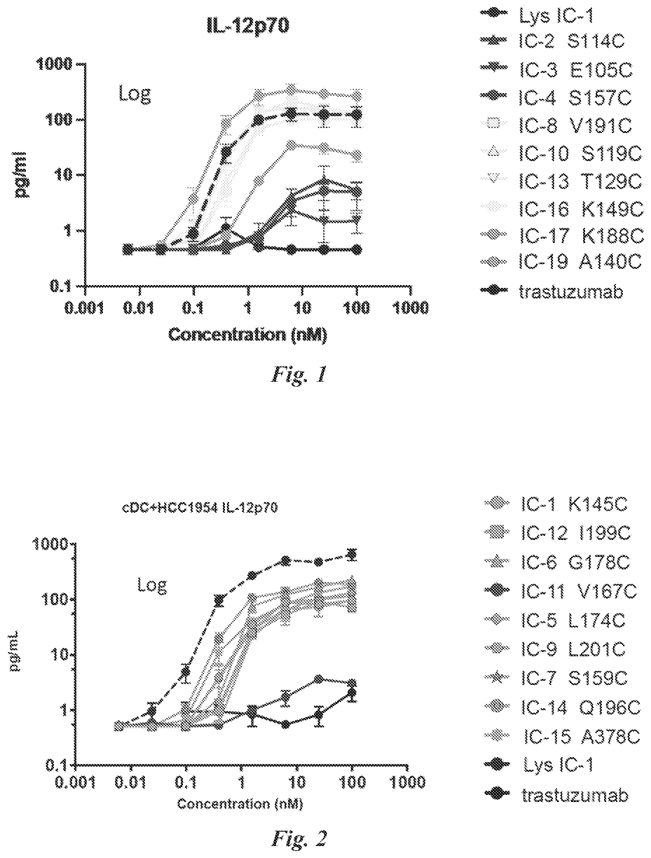

FIG. 1 shows a graph demonstrating IL-12p70 secretion following activation of enriched human cDCs (conventional dendritic cells) freshly isolated from human blood and co-cultured with HCC1954 tumor cells with immunoconjugates Lys IC-1 (Table 11), IC-2, IC-3, IC-4, IC-8, IC-10, IC-13, IC-16, IC-17 and IC-18 (Table 10) and unconjugated antibody, trastuzumab. Logarithmic production of IL-12p70 is plotted at increasing concentrations immunoconjugates and trastuzumab FIG. 2 shows a graph demonstrating IL-12p70 secretion following activation of enriched human cDCs (conventional dendritic cells) freshly isolated from human blood and co-cultured with HCC1954 tumor cells with immunoconjugates IC-1, IC-12, IC-6, IC-11, IC-5, IC-9, IC-7, IC-14, and IC-15 (Table 10), Lys IC-1 (Table 11), and unconjugated antibody, trastuzumab. Logarithmic production of IL-12p70 is plotted at increasing concentrations of immunoconjugates and trastuzumab.

The results in FIGS. 1 and 2 show that certain cysteine-mutant immunoconjugates from Table 10a induce higher levels of IL-12p70 in a cDC assay and thus elicit stronger myeloid action than the comparator amide-linked immunoconjugate, Lys IC-1 from Table 11, and the unconjugated antibody trastuzumab.

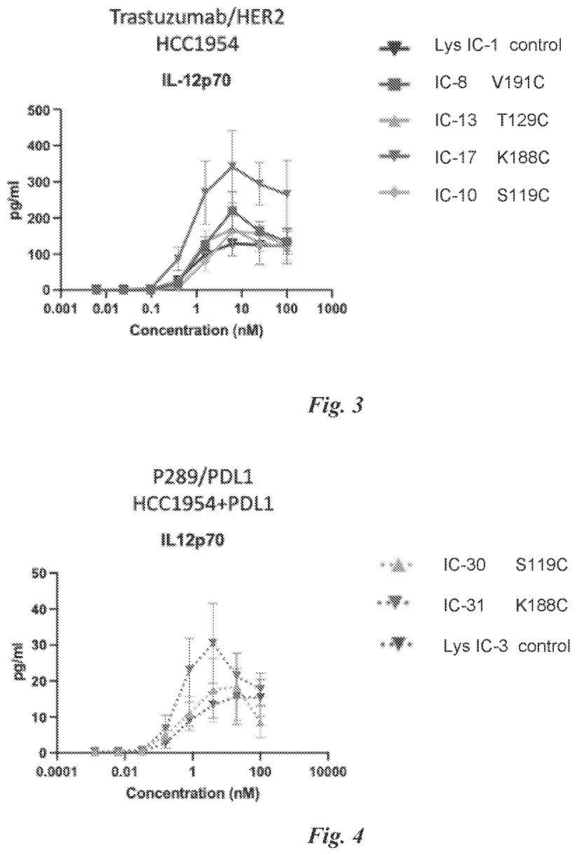

FIG. 3 shows a graph demonstrating IL-12p70 secretion following activation of enriched human cDCs (conventional dendritic cells) freshly isolated from human blood and co-cultured with HCC1954 breast cancer tumor cells with cysteine-mutant, anti-HER2 immunoconjugates IC-8, IC-13, IC-17, and IC-10, and control amide-linked, anti-HER2 conjugate Lys IC-1. The results in FIG. 3 show that certain cysteine-mutant immunoconjugates from Table 10b induce higher levels of IL-12p70 in a breast cancer assay and thus elicit stronger myeloid action than the comparator amide-linked immunoconjugate, Lys IC-1 from Table 11. The K188C mutant IC-17 induced the highest level of IL-12p70.

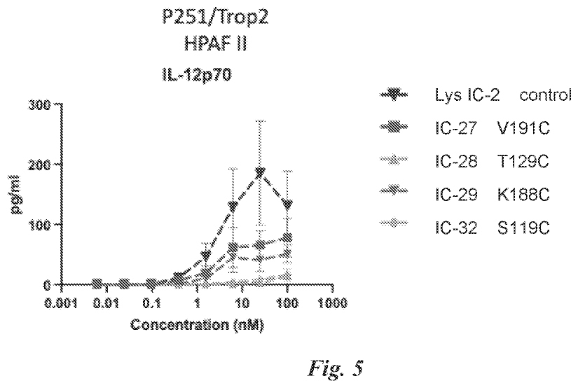

FIG. 4 shows a graph demonstrating IL-12p70 secretion following activation of enriched human cDCs (conventional dendritic cells) freshly isolated from human blood and co-cultured with a modified HCC1954 cell line that overexpresses PD-L1 with cysteine-mutant, anti-PD-L1 immunoconjugates IC-30, IC-31, and control amide-linked, anti-PD-L1 conjugate Lys IC-3. The results in FIG. 4 show that certain cysteine-mutant immunoconjugates from Table 10b induce higher levels of IL-12p70 in a breast cancer assay and thus elicit stronger myeloid action than the comparator amide-linked immunoconjugate, Lys IC-3 from Table 11. The K188C mutant IC-31 induced the highest level of IL-12p70.

FIG. 5 shows a graph demonstrating IL-12p70 secretion following activation of enriched human cDCs (conventional dendritic cells) freshly isolated from human blood and co-cultured with HPAF II pancreatic carcinoma tumor cells with cysteine-mutant, anti-TROP2 immunoconjugates, IC-27, IC-28, IC-29, IC-32, and control amide-linked, anti-TROP2 conjugate Lys IC-2. In this instance, the control amide-linked, anti-TROP2 conjugate Lys IC-2 induced higher levels of IL-12p70 in a breast cancer assay and thus elicited stronger myeloid action than certain cysteine-mutant immunoconjugates from Table 10b.

The results in FIGS. 1-5 show that immunoconjugates of the invention are effective at eliciting myeloid activation, and therefore may be useful for the treatment of cancer.

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of TLR agonist adjuvants linked to different positions on the cysteine-mutant antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug (TLR agonist) loading per cysteine-mutant antibody in the mixture of immunoconjugate compounds is about 2.

A composition of immunoconjugates of the invention can have an average adjuvant to antibody construct ratio (DAR) of about 0.4 to about 10, depending on the number of cysteine mutation sites and the conditions preparing the cysteine-mutant antibody for conjugation, and the conjugation conditions. A skilled artisan will recognize that the number of TLR agonist adjuvants conjugated to the cysteine-mutant antibody construct may vary from immunoconjugate to immunoconjugate in a composition comprising multiple immunoconjugates of the invention and thus the adjuvant to antibody construct (e.g., antibody) ratio can be measured as an average which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC) selected from Table 9.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., cysteine-mutant analogs of atezolizumab, durvalumab, avelumab, biosimilars thereof, or biobetters thereof). In some embodiments, methods for treating breast cancer include administering an immunoconjugate containing an antibody construct that is capable of binding PD-L1 (e.g., cysteine-mutant analogs of atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., cysteine-mutant analogs of atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, Askin's tumor; sarcoma botryoides; chondrosarcoma; Ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); Kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one embodiment, the immunoconjugate is administered to the patient intravenously, intratumorally, or subcutaneously.

The immunoconjugates of the invention may be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein may be used to treat the same types of cancers as naked antibodies approved for treatment such as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The immunoconjugate is administered to a subject in need thereof in any therapeutically effective amount using any suitable dosing regimen. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. In one embodiment, the immunoconjugate is administered to the patient at a dose of about 0.01-20 mg per kg of body weight. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered in a regimen or course of therapy from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. cysteine-mutant analogs of trastuzumab, pertuzumab, biosimilars, or biobetters thereof), PD-L1 (e.g., cysteine-mutant analogs of atezolizumab, durvalumab, avelumab, biosimilars, or biobetters thereof), or TROP2 (e.g. cysteine-mutant analogs of sacituzumab, sacituzumab govetican (TRODELVY@, Immnunomedics, IMMU-132), biosimilars, or biobetters thereof). In some embodiments, methods for treating colon cancer lung cancer, renal cancer, pancreatic cancer, gastric cancer, and esophageal cancer include administering an immunoconjugate containing an antibody construct that is capable of binding CEA, or tumors over-expressing CEA (e.g. labetuzumab, biosimilars, or biobetters thereof).

In some embodiments, the cancer is susceptible to a pro-inflammatory response induced by TLR7 and/or TLR8.

In some embodiments, a therapeutically effective amount of an immunoconjugate is administered to a patient in need to treat cancer wherein the cancer expresses PD-L1, HER2, CEA, or TROP2.

In some embodiments, a therapeutically effective amount of an immunoconjugate is administered to a patient in need to treat cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, or breast cancer. The Merkel cell carcinoma cancer may be metastatic Merkel cell carcinoma. The breast cancer may be triple-negative breast cancer. The esophageal cancer may be gastroesophageal junction adenocarcinoma.

### EXAMPLES

### Example C-1 4-((1-(5-(2-amino-4-(ethoxy(propyl)carbamoyl)-3H-benzo[b]azepin-8-yl)pyrimidin-2-yl)-3-oxo-6,9,12,15,18,21,24,27,30,33-decaoxa-2-azahexatriacontan-36-oyl)oxy)-2,3,5,6-tetrafluorobenzenesulfonic acid, C-1

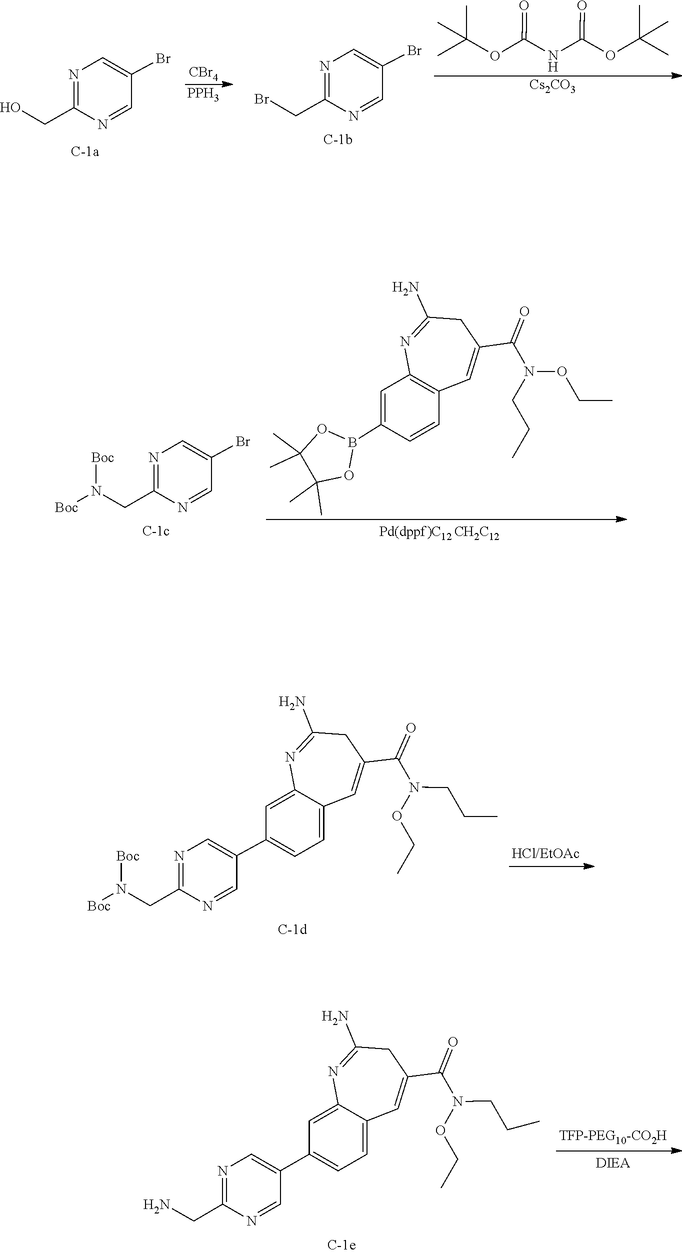

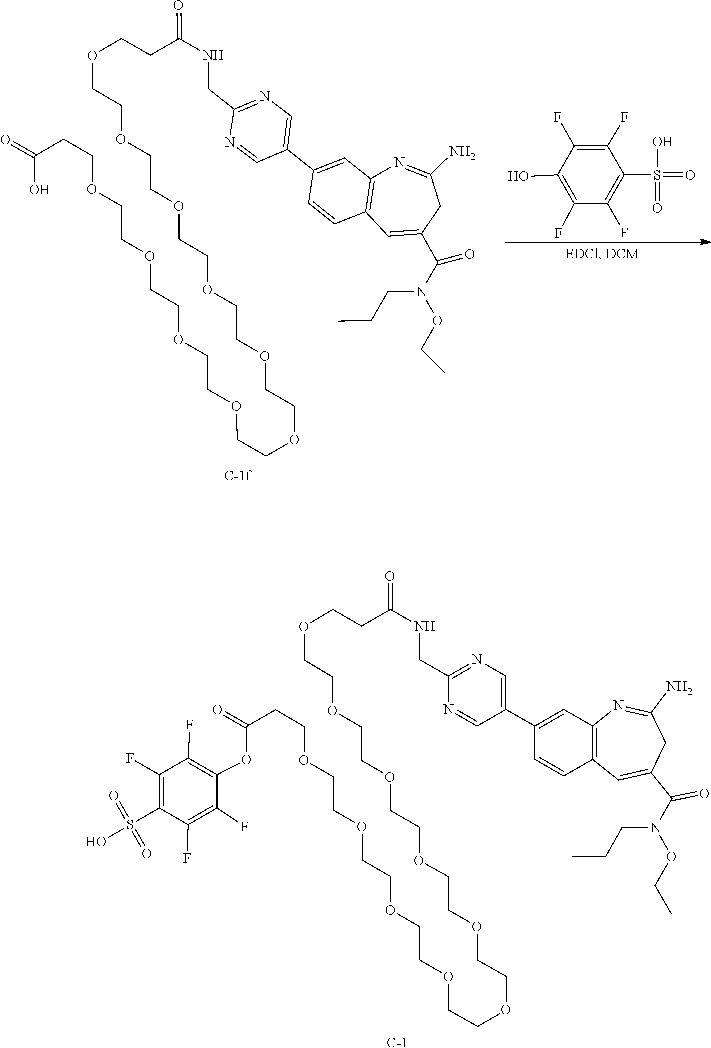

### Preparation of 5-bromo-2-(bromomethyl)pyrimidine, C-1b

To a solution of (5-bromopyrimidin-2-yl)methanol, C-1a (300 mg, 1.59 mmol, 1.0 eq) in THE (10 mL) was added PPh₃ (499 mg, 1.90 mmol, 1.2 eq) and CBr₄ (631 mg, 1.90 mmol, 1.2 eq) in one portion at 0° C. under N₂. The mixture was stirred at 20° C. for 10 hours. Water (10 mL) was added and the aqueous phase was extracted with ethyl acetate (10 mL*3), the combined organic phase was washed with brine (10 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=l/0, 8/1) to afford C-1b (290 mg, 1.15 mmol, 72.4% yield) as white solid. ¹H NMR (400 MHz, CDCl₃) δ8.81 (s, 2H), 4.59 (s, 2H).

### Preparation of tert-butyl N-[(5-bromopyrimidin-2-yl) methyl]-N-tert-butoxycarbonyl-carbamate, C-1c

To a mixture of C-1b (290 mg, 1.15 mmol, 1.0 eq) and tert-butyl N-tert-butoxycarbonylcarbamate (250 mg, 1.15 mmol, 1.0 eq) in DMF (3 mL) was added Cs₂CO₃ (562 mg, 1.73 mmol, 1.5 eq) in portions at 20° C. under N₂, the mixture was stirred at 20° C. for 2.5 hours. Water (5 mL) was added and the aqueous phase was extracted with ethyl acetate (5 mL*3), the combined organic phase was washed with brine (5 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 5/1) to afford C-1c (350 mg, 901 umol, 78.3% yield) as white solid. ¹H NMR (400 MHz, CDCl₃) δ8.74 (s, 2H), 5.01 (s, 2H), 1.48 (s, 18H).

### Preparation of tert-butyl N-[[5-[2-amino-4-[ethoxy(propyl)carbamoyl]-3H-1-benzaze-pin-8-yl]pyrimidin-2-yl]methyl]-N-tert-butoxycarbonyl-carbamate, C-id

To a mixture of C-1c(184 mg, 473 umol, 1.0 eq) and 2-amino-N-ethoxy-N-propyl-8-(4,4,5,5-tetramethyl-1,3,2-dioxaborolan-2-yl)-3H-1-benzazepine-4-carboxamide (195 mg, 474 umol, 1.0 eq) in dioxane (10 mL) and H₂O (2 mL) was added Pd(dppf)Cl₂CH₂Cl₂ (19.3 mg, 23.7 umol, 0.05 eq) and K₂CO₃ (163 mg, 1.18 mmol, 2.5 eq) in one portion under N₂, the mixture was de-gassed and heated to 90° C. for 2 hours under N₂. Dioxane (10 mL) was removed in vacuum and water (20 mL) was added and the aqueous phase was extracted with ethyl acetate (10 mL*3), the combined organic phase was washed with brine (10 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1 to Ethyl acetate/Methanol=10/1) to afford C-id (280 mg, 470.83 umol, 99.35% yield) as gray solid. ¹H NMR (400 MHz, MeOD) δ9.08 (s, 2H), 7.61 (s, 1H), 7.59 (d, J=2.8 Hz, 2H), 7.38 (s, 1H), 5.08 (s, 2H), 3.98 (q, J=7.2 Hz, 2H), 3.76 (t, J=7.2 Hz, 2H), 1.83-1.75 (m, 2H), 1.47 (s, 18H), 1.20 (t, J=7.2 Hz, 3H), 1.02 (t, J=7.2 Hz, 3H).

### Preparation of 2-amino-8-[2-(aminomethyl)pyrimidin-5-yl]-N-ethoxy-N-propyl-3H-1-benzazepine-4-carboxamide, C-1e

To a solution of C-id (20.0 mg, 33.6 umol, 1.0 eq) in EtOAc (5 mL) was added HCl/EtOAc (4 M, 8.41 uL, 1.0 eq) in one portion at 20° C. under N₂, the mixture was stirred at 20° C. for 1 hour. The reaction mixture was concentrated in vacuum. The residue was purified by prep-HPLC (column: Phenomenex Synergi C18 150*25*10 um; mobile phase: [water(0.1% TFA)-ACN]; B %: 1%-30%, 8 min) to afford C-1e (6.2 mg, 9.84 umol, 29.2% yield, 98.8% purity, 2TFA) as white solid. ¹H NMR (400 MHz, MeOD) δ9.22 (s, 2H), 7.82 (d, J=2.0 Hz, 1H), 7.79-7.75 (m, 2H), 7.47 (s, 1H), 4.49 (s, 2H), 4.00 (q, J=7.2 Hz, 2H), 3.78 (t, J=7.2 Hz, 2H), 3.46 (s, 2H), 1.85-1.77 (m, 2H), 1.22 (t, J=7.2 Hz, 3H), 1.03 (t, J=7.2 Hz, 3H). LC/MS [M+H] 395.2 (calculated); LC/MS [M+H] 395.1 (observed).

### Preparation of 3-[2-[2-[2-[2-[2-[2-[2-[2-[2-[3-[[5-[2-amino-4-[ethoxy(propyl)carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, C-1f

To a mixture of C-1e (70 mg, 149 umol, 1.0 eq, 2HCl) and 3-[2-[2-[2-[2-[2-[2-[2-[2-[2-[3-oxo-3-(2,3,5,6-tetrafluorophenoxy)propoxy]ethoxy]ethoxy]ethoxy]ethoxy]ethoxy]ethoxy]ethoxy]ethoxy]ethox y]propanoic acid (127 mg, 179 umol, 1.2 eq) in DMF (0.5 mL) was added diisopropylethylamine, DIEA (77.4 mg, 599 umol, 104 μL, 4.0 eq) in one portion at 25° C. under N₂, the mixture was stirred at 25° C. for 0.5 hour. The reaction mixture was filtered and filtrate was purified by prep-HPLC (column: Phenomenex luna C18 80*40 mm*3 micron (μm); mobile phase: [water(0.04% HCl)-ACN]; B %: 12%-39%, 5.5 min) to afford C-if (50.0 mg, 53.4 umol, 35.7% yield) as yellow oil. ¹H NMR (400 MHz, MeOD) δ9.14 (s, 2H), 7.86-7.81 (m, 1H), 7.78-7.74 (m, 2H), 7.48 (s, 1H), 4.72 (s, 2H), 4.00 (q, J=7.2 Hz, 2H), 3.85-3.71 (m, 8H), 3.69-3.58 (m, 38H), 3.47 (s, 2H), 2.62 (t, J=6.0 Hz, 2H), 2.55 (t, J=6.4 Hz, 2H), 1.85-1.76 (m, 2H), 1.23 (t, J=7.2 Hz, 3H), 1.03 (t, J=7.2 Hz, 3H).

### Preparation of C-1

To a mixture of C-1f (60 mg, 61.7 umol, 1.0 eq, HCl) and (2,3,5,6-tetrafluoro-4-hydroxy-phenyl)sulfonyloxy sodium (99.3 mg, 370 umol, 6.0 eq) in dichloromethane, DCM (2 mL) and dimethylacetamide, DMA (0.5 mL) was added 1-Ethyl-3-(3-dimethylaminopropyl)carbodiimide, EDCI, CAS Reg. No. 1892-57-5 (71.0 mg, 370 umol, 6.0 eq) in one portion at 25° C. under N₂, the mixture was stirred at 25° C. for 1 hours. The reaction mixture was filtered and the filtrate was purified by prep-HPLC (column: Phenomenex Synergi C18 150*25*10 um; mobile phase: [water(0.1% TFA)-ACN]; B %: 20%-45%, 8 min) to afford C-1 (38.0 mg, 30.5 umol, 49.3% yield, 93.3% purity) as yellow oil. ¹H NMR (400 MHz, MeOD) δ9.11 (s, 2H), 7.83-7.79 (m, 1H), 7.77 (s, 1H), 7.76-7.71 (m, 1H), 7.47 (s, 1H), 4.71 (s, 2H), 4.00 (q, J=7.2 Hz, 2H), 3.88 (t, J=5.6 Hz, 2H), 3.85-3.75 (m, 5H), 3.70-3.57 (m, 38H), 3.47 (s, 2H), 2.99 (t, J=6.0 Hz, 2H), 2.62 (t, J=4 Hz, 2H), 1.85-1.75 (m, 2H), 1.23 (t, J=7.2 Hz, 3H), 1.02 (t, J=7.2 Hz, 3H). LC/MS [M+H] 1163.3 (calculated); LC/MS [M+H] 1163.3 (observed).

### Example C-2 2,3,5,6-tetrafluorophenyl 1-(5-(2-amino-4-(ethoxy(propyl)carbamoyl)-3H-benzo[b]azepin-8-yl)pyrimidin-2-yl)-3-oxo-6,9,12,15,18,21,24,27,30,33-decaoxa-2-azahexatriacontan-36-oate, C-2

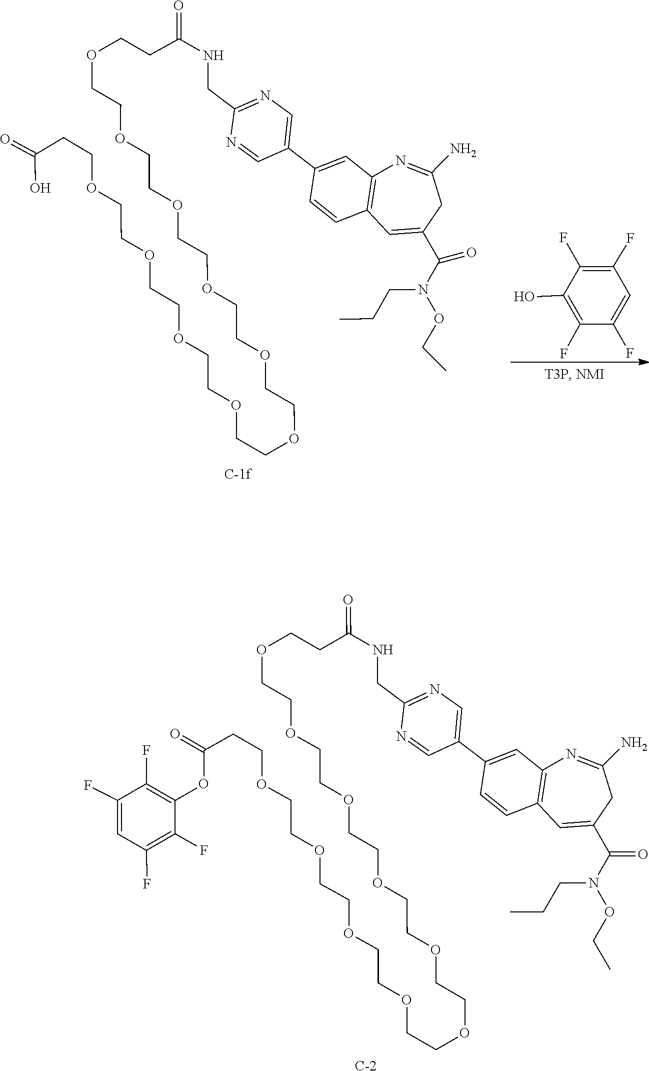

Following the procedures of Example C-1, to a solution of 3-[2-[2-[2-[2-[2-[2-[2-[2-[2-[3-[[5-[2-amino-4-[ethoxy(propyl)carbamoyl]-3H-1-benzazepin-8-yl]pyrimidin-2-yl]methylamino]-3-oxo-propoxy]ethoxy]ethoxy]ethoxy]ethoxy]ethoxy]ethoxy]ethoxy]ethoxy]ethoxy]propanoic acid, C-lf (5.00 g, 5.35 mmol, 1.00 equiv.) in 50 ml DCM were added 2,3,5,6-tetrafluorophenol (1.77 g, 10.7 mmol, 2.00 equiv.), Propanephosphonic acid anhydride (PPAA, T3P), CAS Reg. No. 68957-94-8 (50 wt % solution in MeCN, 17.0 g solution, 26.8 mmol, 5.00 equiv.) and N-methylimidazole, NMI (2.15 mL, 26.8 mmol, 5.00 equiv.) sequentially. The mixture was stirred at 20° C. for 2 h and then diluted with 20% aq NaCl (50 mL). The aqueous layer was extracted with DCM (25 mL) and the combined organic layers washed with water (25 mL), dried (Na2SO4), filtered, and concentrated in vacuo to obtain crude C-2 in the form of dark brown oil. The material was loaded onto a Biotage column (250 mL 7.5 mM HCl in MeCN/water 2:8, v/v) and purified using a gradient step (20 column volumes MeCN/water 2:8, then 15 column volumes MeCN/water 3:7). The desired fractions were combined and then extracted (2×300 mL DCM) and concentrated in vacuo to afford pure C-2 (5.34 g, 55.6 wt % purity by qNMR, 56% yield) in the form of dark yellow oil which was stored at −20° C. under nitrogen before it was diluted with DMA to make a 20 mM solution of C-2 LC/MS [M+H] 1083.1 (calculated); LC/MS [M+H] 1083.1 (observed).

### Example TLR-L-1 2-amino-8-(2-(38-(2,5-dioxo-2,5-dihydro-1H-pyrrol-1-yl)-3,37-dioxo-6,9,12,15,18,21,24,27,30,33-decaoxa-2,36-diazaoctatriacontyl)pyrimidin-5-yl)-N-ethoxy-N-propyl-3H-benzo[b]azepine-4-carboxamide, TLR-L-1

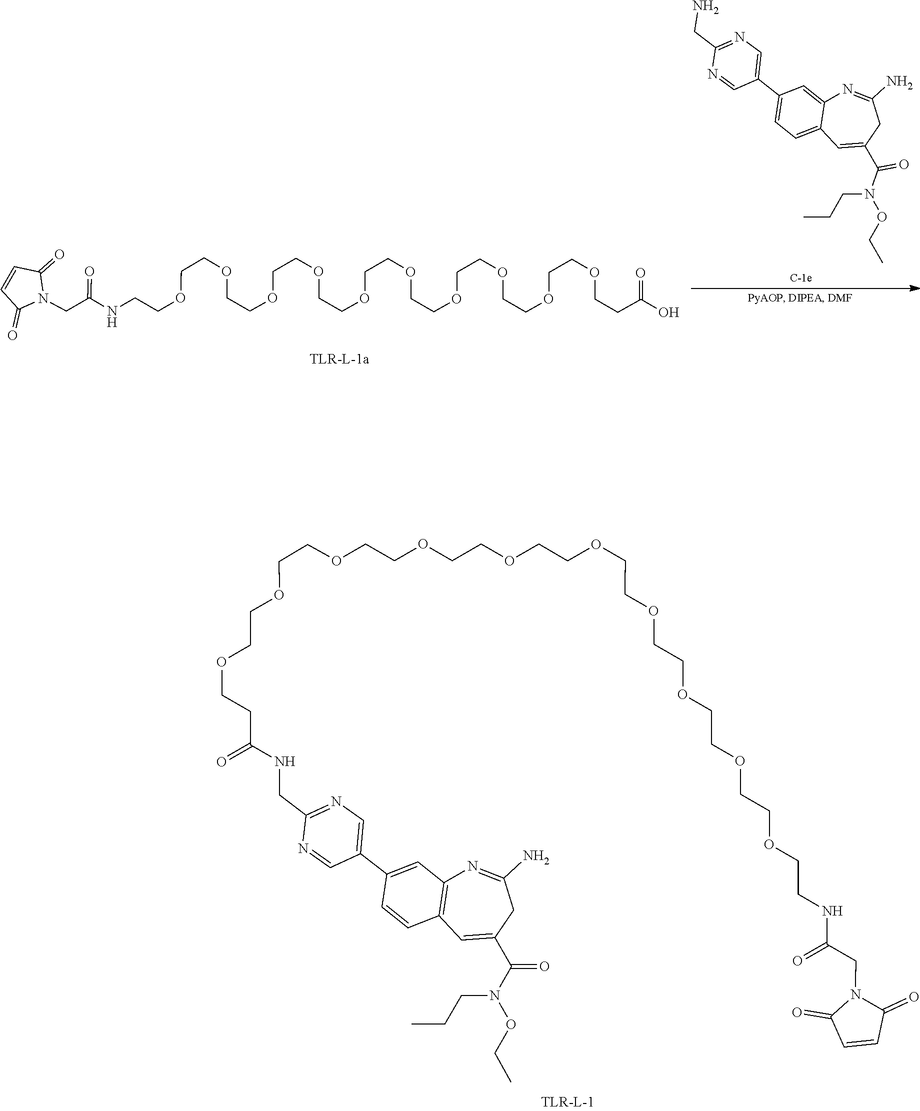 [SMILES: O=C(O)CCOCCOCCOCCOCCOCCOCCOCCOCCOCCOCCNC(=O)CN1C(=O)C=CC1=O ; CCCN(OCC)C(=O)C1=Cc2ccc(-c3cnc(CN)nc3)cc2N=C(N)C1 ; CCCN(OCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1]

2-Amino-8-(2-(aminomethyl)pyrimidin-5-yl)-N-ethoxy-N-propyl-3H-benzo[b]azepine-4-carboxamide, C-1e (0.0283 g, 0.072 mmol, 1 eq.) and 1-(2,5-dioxo-2,5-dihydro-1H-pyrrol-1-yl)-2-oxo-6,9,12,15,18,21,24,27,30,33-decaoxa-3-azahexatriacontan-36-oic acid, TLR-L-la (0.0478 g, 0.072 mmol, 1 eq.) were dissolved in dimethylformamide, DMF. Diisopropylethylamine, DIPEA (0.075 mol, 0.43 mmol, 6 eq.) was added, followed by ((7-Azabenzotriazol-1-yloxy)tripyrrolidinophosphoniurn hexafluorophosphate), PyAOP, CAS Reg. No. 156311-83-0 (0.091 g, 0.18 mmol, 2.4 eq.). The reaction was stirred at room temperature, then concentrated and purified by RP-HPLC to give TLR-L-1 (0.0346 g, 0.033 mmol, 46%). LC/MS [M+H] 1043.53 (calculated); LC/MS [M+H] 1043.84 (observed).

### Example 201 Preparation of Anti-HER2 Antibody with Specific Cysteine (Cys) Mutations

Preparation of anti-HER2 antibodies, e.g., trastuzumab, with site-specific cysteine mutations are described in U.S. Pat. No. 10,973,826, WO 2014/124316, and WO 2015/138615, each of which is incorporated by reference herein. DNA encoding variable regions of the heavy and light chains of an anti-HER2 antibody, e.g., trastuzumab, are chemically synthesized and cloned into two mammalian expression vectors, pOG-HC and pOG-LC, that contain constant regions of human IgG1 and human kappa light chain. Vectors contain a CMV promoter and a signal sequence. Oligonucleotide directed mutagenesis was employed to prepare Cys mutant constructs of the anti-HER2 antibody, and the sequences of Cys mutant constructs were confirmed by DNA sequencing. For example, cysteine can be introduced at one or more of the following positions (all positions by EU numbering) in an anti-HER2 antibody: (a) positions S157, L174, G178, S119, V167, I199, Q196, A378 and A140 of the antibody heavy chain, and (b) positions K145, S114, E105, S159, V191, L201, T129, K149 and K188 of the antibody light chain. For example, cysteine can be introduced at position S157 (Table 3) of the heavy chain resulting in an anti-HER2 mAb, which has a light chain sequence of SEQ ID NO: 21 and a heavy chain sequence of SEQ ID NO: 33 (Table 4).

Cys mutants of the anti-HER2 antibody may be expressed in 293 Freestyle™ cells by co-transfecting heavy chain and light chain plasmids using transient transfection methods as described (Meissner, et al., (2001) Biotechnol Bioeng. 75:197-203). The expressed antibodies are purified from the cell supernatants by standard Protein A affinity chromatography.

Similar methods are used to clone the variable regions of the heavy chain and light chain of trastuzumab into two vectors for expression in CHO cells. The heavy chain vector encodes the constant region of the human IgG1 antibody, includes a signal peptide, a CMV promoter to drive expression of the heavy chain, and appropriate signal and selection sequences for stable transfection into CHO cells. The light chain vector encodes the constant region of the human kappa light chain, and includes a signal peptide, a CMV promoter to drive expression of the light chain, and appropriate signal and selection sequences for stable transfection into CHO cells. To produce antibodies, a heavy chain vector and a light chain vector are co-transfected into a CHO cell line. Cells undergo selection, and stably transfected cells are then cultured under conditions optimized for antibody production. Antibodies are purified from the cell supernatants by standard Protein A affinity chromatography.

### Reduction, Re-Oxidation and Conjugation of Cys Mutant Anti-HER2 Antibodies to TLR7 Agonists

TLR agonists of the invention comprising a linker, such as (TLR-L), are conjugated to Cysteine (Cys) residues engineered into an antibody using methods described in Junutula J R, et al., Nature Biotechnology 26:925-932 (2008), and Example 202. Because engineered Cys residues in antibodies expressed in mammalian cells are modified by adducts (disulfides) such as glutathione (GSH) and/or cysteine during biosynthesis, the modified Cys as initially expressed is unreactive to thiol reactive reagents such as maleimido or bromo-acetamide or iodo-acetamide groups. To conjugate engineered Cys residues, glutathione or cysteine adducts are removed by reducing disulfides, which generally entails reducing all disulfides in the expressed antibody. Reduction is accomplished by first exposing the antibody to a reducing agent such as dithiothreitol (DTT) or TCEP followed by re-oxidation of all native disulfide bonds of the antibody to restore and/or stabilize the functional antibody structure. Accordingly, in order to reduce native disulfide bonds and disulfide bond between the cysteine or GSH adducts of engineered Cys residue(s), freshly prepared DTT is added to previously purified Cys mutants of trastuzumab, to a final concentration of 10 mM or 20 mM. After antibody incubation with DTT at 37° C. for 1 hour, mixtures are dialyzed against PBS for three days with daily buffer exchange to remove reducing agent and byproducts, such as DTT, and re-oxidize native disulfide bonds. The re-oxidation process is monitored by reverse-phase HPLC, which is able to separate antibody tetramer from individual heavy and light chain molecules. Reactions are analyzed on a PRLP-S 4000A column (50 mm×2.1 mm, Agilent) heated to 80° C. and column elution is carried out by a linear gradient of 30-60% acetonitrile in water containing 0.1% TFA at a flow rate of 1.5 ml/min. The elution of proteins from the column is monitored at 280 nm. Dialysis is allowed to continue until reoxidation is complete. Reoxidation restores intra-chain and interchain disulfides, while dialysis allows cysteines and glutathiones connected to the newly-introduced Cys residue(s) to dialyze away.

After re-oxidation is complete or near-complete, TLR agonist linker maleimide-containing intermediate compounds (TLR-L) are added to re-oxidized antibodies in PBS buffer (pH 7.2) at ratios of typically 1.5:1, 2:1, or 5:1 to engineered Cys, and incubations are carried out for about 1 hour. Typically, excess free TLR-L is removed by purification over Protein A resin by standard methods followed by buffer exchange into PBS.

Alternatively, Cys mutants of anti-HER2 antibody, e.g., trastuzumab, are reduced and re-oxidized using an on-resin method. Protein A Sepharose beads (1 ml per 10 mg antibody) are equilibrated in PBS (no calcium or magnesium salts) and then added to an antibody sample in batch mode. A stock of 0.5 M cysteine is prepared by dissolving 850 mg of cysteine HCl in 10 ml of a solution prepared by adding 3.4 g of NaOH to 250 ml of 0.5 M sodium phosphate pH 8.0 and then 20 mM cysteine is added to the antibody/bead slurry, and mixed gently at room temperature for 30-60 minutes. Beads are loaded to a gravity column and washed with 50 bed volumes of PBS in less than 30 minutes, then the column is capped with beads resuspended in one bed volume of PBS. To modulate the rate of re-oxidation, 50 nM to 1 μM (micromolar) copper chloride is optionally added. The re-oxidation progress is monitored by removing a small test sample of the resin, eluting in IgG Elution buffer (Thermo), and analyzing by RP-HPLC as described above. Once re-oxidation progresses to desired completeness, conjugation is initiated immediately by addition of 2-3 molar excess of TLR-L compound over engineered cysteines, and allowing the mixture to react for 5-10 minutes at room temperature before the column was washed with at least 20 column volumes of PBS. Antibody conjugates are eluted with IgG elution buffer and neutralized with 0.1 volumes 0.5 M sodium phosphate pH 8.0 and buffer exchanged to PBS. Alternatively, instead of initiating conjugation with antibody on the resin, the column is washed with at least 20 column volumes of PBS, and antibody is eluted with IgG elution buffer and neutralized with buffer pH 8.0. Antibodies are then either used for conjugation reactions or flash frozen for future use.

### Example 202 Preparation of Cysteine Mutant Immunoconjugates (IC)

For preparation of Cysteine mutant-based conjugation, the antibody is buffer exchanged into phosphate-buffered saline (PBS) containing 2 mM ethylenediaminetetraacetic acid (EDTA) at pH 7.2 using Zeba™ Spin Desalting Columns (Thermo Fisher Scientific). The concentration of the buffer-exchanged antibody was adjusted to approximately 5 to 25 mg/ml and sterile filtered. As a first step, a 20 to 40 fold molar excess of reducing agent such as dithiothreitol (DTT) or tris-(2-carboxyethyl)phosphine (TCEP) was added to the antibody to reduce all disulfide bonds and to remove glutathione and/or Cysteine adducts. The reduction was done at 30° C. or 37° C. for 30 min to 2 hours. Excess reducing reagent was removed using Zeba Spin Desalting Columns. The native disulfides were restored by re-oxidizing the Ab using 20-40 fold molar excess of dehydroascorbic acid (dhAA) for 60 min to 2 hours at room temperature. After the re-oxidation is complete, the maleimide containing linker-payload is added to the re-oxidized antibody in 5-12 fold molar excess at room temperature for 1 hour. Excess linker-payload was removed by buffer exchanging into an appropriate formulation buffer using Zeba Spin Desalting Columns. After conjugation, the resulting succinimide ring of the IC may be hydrolyzed to impart greater stability (Zheng, K. et al (2019) J Pharm Sci, 108(1):133-141)

Following conjugation, to potentially remove unreacted TLR-L and/or higher-molecular weight aggregate, the IC may be purified further using size exclusion chromatography, hydrophobic interaction chromatography, ion exchange chromatography, chromatofocusing, ultrafiltration, centrifugal ultrafiltration, tangential flow filtration, and combinations thereof.

For conjugation, the antibody may be dissolved in a aqueous buffer system known in the art that will not adversely impact the stability or antigen-binding specificity of the antibody. Phosphate buffered saline may be used. The TLR-L is dissolved in a solvent system comprising at least one polar aprotic solvent as described elsewhere herein. In some such aspects, the TLR-L is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the TLR-L is dissolved in DMSO (dimethylsulfoxide), DMA (dimethylacetamide) or acetonitrile, or another suitable dipolar aprotic solvent.

Alternatively in the conjugation reaction, an equivalent excess of TLR-L solution may be diluted and combined with antibody solution. The TLR-L solution may suitably be diluted with at least one polar aprotic solvent and at least one polar protic solvent, examples of which include water, methanol, ethanol, n-propanol, and acetic acid. The molar equivalents of TLR-L to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the TLR-L, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 203 Preparation of Comparator Amide-Linked Immunoconjugates

To prepare a lysine conjugated Immunoconjugate, such comparator Lys IC-1 from Table 11, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO) or Zeba™ Spin Desalting Columns (Thermo Fisher Scientific). The eluates are then each adjusted to a concentration of about 1-10 mg/ml of buffer-exchanged antibody using the buffer and then sterile filtered. The antibody is pre-warned to 20-30° C. and rapidly mixed with 2-20 (e.g., 7-10) molar equivalents of a TLR agonist-linker comparator compound with an active ester, such as 2,3,5,6-tetrafluorophenyl ester or 4-sulfo, 2,3,5,6-tetrafluorophenyl ester from Table 9 to form an amide linkage to the antibody. The reaction is allowed to proceed for about 16 hours at 30° C. and the immunoconjugate (IC) is separated from reactants by running over two successive G-25 desalting columns equilibrated in phosphate buffered saline (PBS) at pH 7.2 to provide the Immunoconjugate (IC) of Table 2. Adjuvant-antibody ratio (DAR) is determined by liquid chromatography mass spectrometry analysis using a C₄ reverse phase column on an ACQUITY™ UPLC H-class (Waters Corporation, Milford, MA) connected to a XEVO™ G2-XS TOF mass spectrometer (Waters Corporation).

Alternatively, the active-ester TLR agonist-linker (TLR-L) compound of formulas a-f is dissolved in dimethylsulfoxide (DMSO) or dimethylacetamide (DMA) to a concentration of 5 to 20 mM. For conjugation, the antibody is mixed with 4 to 20 molar equivalents of TLR-L. In some instances, additional DMA or DMSO up to 20% (v/v), is added to improve the solubility of TLR-L in the conjugation buffer. The reaction is allowed to proceed for approximately 30 min to 4 hours at 20° C. or 30° C. or 37° C. The resulting conjugate is purified away from the unreacted BBI-L using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C₄ reverse phase column on an ACQUIIY™ UPLC H-class (Waters Corporation, Milford, MA) connected to a XVO™ G2-XS TOF mass spectrometer (Waters Corporation).

### Example 204 Assessment of Immunoconjugate Activity In Vitro

This example shows that Immunoconjugates of the invention are effective at eliciting myeloid activation, such as in dendritic cells, and therefore are useful for the treatment of cancer.

Isolation of Human Conventional Dendritic Cells: Human conventional dendritic cells (cDCs) were negatively selected from human peripheral blood obtained from healthy blood donors (Stanford Blood Center, Palo Alto, California) by density gradient centrifugation. Briefly, cells are first enriched by using a ROSETTESEP™ Human CD3 Depletion Cocktail (Stem Cell Technologies, Vancouver, Canada) to remove T cells from the cell preparation. cDCs are then further enriched via negative selection using an EASYSEP™ Human Myeloid DC Enrichment Kit (Stem Cell Technologies).

cDC Activation Assay: 8×10⁴ APCs were co-cultured with tumor cells expressing the ISAC target antigen at a 10:1 effector (cDC) to target (tumor cell) ratio. Cells were incubated in 96-well plates (Corning, Corning, NY) containing RPMI-1640 medium supplemented with 10% FBS, and where indicated, various concentrations of the indicated immunoconjugate of the invention (as prepared according to the example above). Following overnight incubation of about 18 hours, cell-free supernatants were collected and analyzed for cytokine secretion (including TNFα and/or IL-12p70) using a BioLegend LEGENDPLEX cytokine bead array.

Activation of myeloid cell types can be measured using various screen assays in addition to the assay described in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, conventional Dendritic Cells (cDCs) isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a M((IL4/IL13), M2c MΦ(IL10/TGFb), GM-CSF/IL6 MDSCs and tumor-educated monocytes (TEM). TEM differentiation can be performed using tumor-conditioned media (e.g. 786.0, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the ISAC may bind to via the CDR region of the antibody. Following incubation for 18-48 hours, activation may be assessed by upregulation of cell surface co-stimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate comprising a cysteine-mutant antibody covalently attached to one or more TLR agonist moieties by a linker.

2. The immunoconjugate of claim 1 wherein the cysteine-mutant antibody comprises a cysteine mutation in the hinge region.

3. The immunoconjugate of claim 1 wherein the cysteine-mutant antibody comprises a cysteine mutation selected from the group consisting of: K145C, S114C, E105C, S157C, L174C, G178C, T159C, V191C, L201C, S119C, V167C, I199C, T129C, Q196C, A378C, K149C, K188C, and A140C, numbered according to the EU format.

4. The immunoconjugate of claim 3 wherein the cysteine-mutant antibody comprises a light chain cysteine mutation in a sequence selected from the group consisting of:

| Sequence: | mutant site | SEQ ID NO: |
|---|---|---|
| YPREACVQWKV | LC K145C | 1 |
| TVAAPCVFIFP | LC S114C | 2 |
| QGTKVCIKRTV | LC E105C | 3 |
| LQSGNCQESVT | LC S159C | 7 |
| YEKHKCYACEV | LC V191C | 8 |
| VTHQGCSSPVT | LC L201C | 9 |
| QLKSGCASVVC | LC T129C | 13 |
| AKVQWCVDNAL | LC K149C | 16 |
| KADYECHKVYA | LC K188C | 17. |

5. The immunoconjugate of claim 4 wherein the heavy chain of the cysteine-mutant antibody has the sequence of SEQ ID NO: 20.

6. The immunoconjugate of claim 4 wherein the light chain of the cysteine-mutant antibody is selected from SEQ ID NO: 24, 25, 26, 27, 28, 29, 30, 31, and 32.

7. The immunoconjugate of claim 3 wherein the cysteine-mutant antibody comprises a heavy chain cysteine mutation in a sequence selected from the group consisting of:

| Sequence: | mutant site | SEQ ID NO: |
|---|---|---|
| EPVTVCWNSGA | HC S157C | 4 |
| TFPAVCQSSGL | HC L174C | 5 |
| VLQSSCLYSLS | HC G178C | 6 |
| TVSSACTKGPS | HC S119C | 10 |
| ALTSGCHTFPA | HC V167C | 11 |
| GTQTYCCNVNH | HC I199C | 12 |
| SSLGTCTYICN | HC Q196C | 14 |
| YPSDICVEWES | HC A378C | 15 |
| TSGGTCALGCL | HC A140C | 18. |

8. The immunoconjugate of claim 7 wherein the light chain of the cysteine-mutant antibody has the sequence of SEQ ID NO: 21.

9. The immunoconjugate of claim 7 wherein the heavy chain of the cysteine-mutant antibody is selected from SEQ ID NO: 33, 34, 35, 36, 37, 38, 39, 40, and 41.

10. The immunoconjugate of claim 3 wherein the cysteine-mutant antibody comprises a light chain cysteine mutation in a sequence selected from the group consisting of:

| Sequence: | mutant site | SEQ ID NO: |
|---|---|---|
| KADYECHKVYA | LC K188C | 17 |
| YEKHKCYACEV | LC V191C | 8 |
| QLKSGCASVVC | LC T129C | 13. |

11. The immunoconjugate of claim 10 wherein the heavy chain of the cysteine-mutant antibody has the sequence of SEQ ID NO: 22.

12. The immunoconjugate of claim 10 wherein the light chain of the cysteine-mutant antibody is selected from SEQ ID NO: 42, 43, and 44.

13. The immunoconjugate of claim 3 wherein the cysteine-mutant antibody comprises a heavy chain cysteine mutation in the sequence:

| Sequence: | mutant site | SEQ ID NO: |
|---|---|---|
| TVSSACTKGPS | HC S119C | 19. |

14. The immunoconjugate of claim 13 wherein the light chain of the cysteine-mutant antibody has the sequence of SEQ ID NO: 23.

15. The immunoconjugate of claim 13 wherein the heavy chain of the cysteine-mutant antibody has the sequence of SEQ ID NO:45.

16. The immunoconjugate of claim 1 wherein the cysteine-mutant antibody binds to an antigen selected from PD-L1, HER2, CEA, and TROP2.

17. The immunoconjugate of claim 16 wherein the cysteine-mutant antibody binds to HER2 and comprises:
CDR-L1 comprising an amino acid sequence of SEQ ID NO:47, CDR-L2 comprising an amino acid sequence of SEQ ID NO:49, CDR-L3 comprising an amino acid sequence of SEQ ID NO:51, CDR-H1 comprising an amino acid sequence of SEQ ID NO:54, CDR-H2 comprising an amino acid sequence of SEQ ID NO:56, and CDR-H3 comprising an amino acid sequence of SEQ ID NO:58.

18. The immunoconjugate of claim 16 wherein the cysteine-mutant antibody binds to TROP2 and comprises:
a) CDR-L1 comprising an amino acid sequence of SEQ ID NO:61, CDR-L2 comprising an amino acid sequence of SEQ ID NO:63, CDR-L3 comprising an amino acid sequence of SEQ ID NO:65, CDR-H1 comprising an amino acid sequence of SEQ ID NO:68, CDR-H2 comprising an amino acid sequence of SEQ ID NO:70, and CDR-H3 comprising an amino acid sequence of SEQ ID NO:72; or
b) CDR-L1 comprising an amino acid sequence of SEQ ID NO:75, CDR-L2 comprising an amino acid sequence of SEQ ID NO:77, CDR-L3 comprising an amino acid sequence of SEQ ID NO:79, CDR-H1 comprising an amino acid sequence of SEQ ID NO:82, CDR-H2 comprising an amino acid sequence of SEQ ID NO:84, and CDR-H3 comprising an amino acid sequence of SEQ ID NO:86.

19. The immunoconjugate of claim 16 wherein the cysteine-mutant antibody binds to PD-L1 and comprises:
CDR-L1 comprising an amino acid sequence of SEQ ID NO:89, CDR-L2 comprising an amino acid sequence of SEQ ID NO:91, CDR-L3 comprising an amino acid sequence of SEQ ID NO:93, CDR-H1 comprising an amino acid sequence of SEQ ID NO:96, CDR-H2 comprising an amino acid sequence of SEQ ID NO:98, and CDR-H3 comprising an amino acid sequence of SEQ ID NO:100.

20. The immunoconjugate of claim 16 wherein the cysteine-mutant antibody binds to CEA and comprises:
CDR-L1 comprising an amino acid sequence of SEQ ID NO:103, CDR-L2 comprising an amino acid sequence of SEQ ID NO:105, CDR-L3 comprising an amino acid sequence of SEQ ID NO:107, CDR-H1 comprising an amino acid sequence of SEQ ID NO:110, CDR-H2 comprising an amino acid sequence of SEQ ID NO:112, and CDR-H3 comprising an amino acid sequence of SEQ ID NO:114.

21. The immunoconjugate of claim 1, having Formula I:
Ab-[L-D]ₚ I
or a pharmaceutically acceptable salt thereof,
wherein:
Ab is the cysteine-mutant antibody;
p is an integer from 1 to 8;
L is the linker;
D is the TLR agonist moiety selected from formulas a-f: 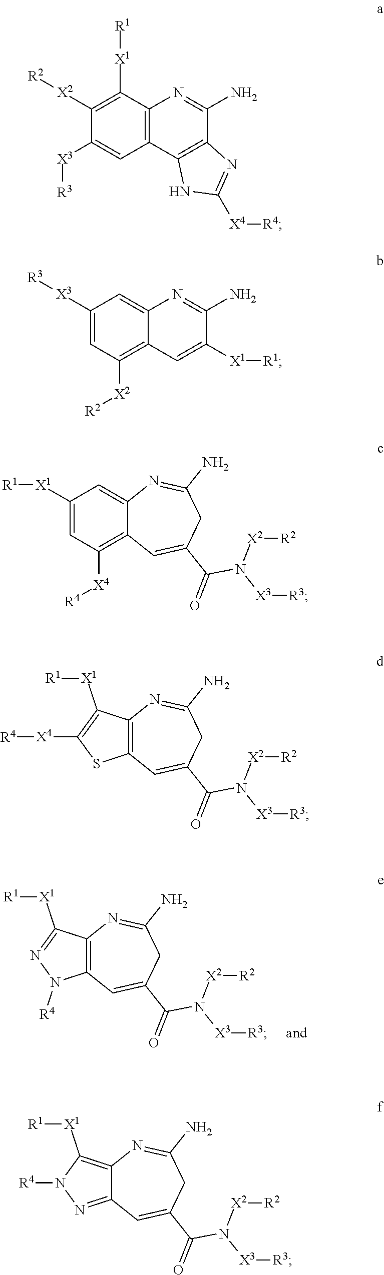
X¹, X², X³ and X⁴ are independently selected from the group consisting of a bond, C(═O), C(═O)N(R⁵), O, N(R⁵), S, S(O)₂, and S(O)₂N(R⁵);
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryldiyl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-C(═O)—(C₁-C₁₂ alkyldiyl)-N(R⁵)—*;
—(C₂-C₉ heterocyclyl)-NR⁵—C(═NR^{5a})NR⁵—*;
—(C₂-C₉ heterocyclyl)-NR⁵—(C₆-C₂₀ aryldiyl)-(C₁-C₁₂ alkyldiyl)-N(R⁵)—*;
—(C₂-C₉ heterocyclyl)-(C₆-C₂₀ aryldiyl)-*;
—(C₁-C₂₀ heteroaryl);
—(C₁-C₂₀ heteroaryldiyl)-*;
—(C₁-C₂₀ heteroaryldiyl)-(C₁-C₁₂ alkyldiyl)-N(R⁵)—*;
—(C₁-C₂₀ heteroaryldiyl)-(C₁-C₁₂ alkyldiyl)-N(R⁵)₂;
—(C₁-C₂₀ heteroaryldiyl)-NR⁵—C(═NR^{5a})N(R⁵)—*;
—(C₁-C₂₀ heteroaryldiyl)-N(R⁵)C(═O)—(C₁-C₁₂ alkyldiyl)-N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-*;
—C(═O)N(R⁵)—(C₁-C₁₂ alkyldiyl)-C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₅ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—N(R⁵)CO₂(R⁵)—*;
—NR⁵C(═NR^{5a})N(R⁵)₂;
—NR⁵C(═NR^{5a})N(R⁵)—*;
—NR⁵C(═NR^{5a})R⁵;
—N(R⁵)C(═O)—(C₁-C₁₂ alkyldiyl)-N(R⁵)—*;
—N(R⁵)—(C₂-C₅ heteroaryl);
—N(R⁵)—S(═O)₂—(C₁-C₂ alkyl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—OC(═O)N(R⁵)₂;
—OC(═O)N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
R⁵ is selected from the group consisting of H, C₆-C₂₀ aryl, C₃-C₁₂ carbocyclyl, C₂-C₂₀ heterocyclyl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)—PEG-;
—C(═O)—PEG-C(═O)N(R⁶)—(C₁-C₁₂ alkyldiyl)-C(═O)-Gluc-;
—C(═O)—PEG-O—;
—C(═O)—PEG-O—C(═O)—;
—C(═O)—PEG-C(═O)—;
—C(═O)—PEG-C(═O)—PEP-;
—C(═O)—PEG-N(R⁶)—;
—C(═O)—PEG-N(R⁶)—C(═O)—;
—C(═O)—PEG-N(R⁶)—PEG-C(═O)—PEP-;
—C(═O)—PEG-N+(R⁶)₂-PEG-C(═O)—PEP-;
—C(═O)—PEG-C(═O)—PEP-N(R⁶)—(C₁-C₁₂ alkyldiyl)-;
—C(═O)—PEG-C(═O)—PEP-N(R⁶)—(C₁-C₁₂ alkyldiyl)N(R⁶)C(═O)—(C₂-C₅ monoheterocyclyldiyl)-;
—C(═O)—PEG-SS—(C₁-C₁₂ alkyldiyl)-OC(═O)—;
—C(═O)—PEG-SS—(C₁-C₁₂ alkyldiyl)-C(═O)—;
—C(═O)—(C₁-C₁₂ alkyldiyl)-C(═O)—PEP-;
—C(═O)—(C₁-C₁₂ alkyldiyl)-C(═O)—PEP-N(R⁶)—(C₁-C₁₂ alkyldiyl)-;
—C(═O)—(C₁-C₁₂ alkyldiyl)-C(═O)—PEP-N(R⁶)—(C₁-C₁₂ alkyldiyl)-N(R⁵)—C(═O);
—C(═O)—(C₁-C₁₂ alkyldiyl)-C(═O)—PEP-N(R⁶)—(C₁-C₁₂ alkyldiyl)-N(R⁶)C(═O)—(C₂-C₅ monoheterocyclyldiyl)-;
succinimidyl-(CH₂)ₘ—C(═O)N(R⁶)—PEG-;
succinimidyl-(CH₂)ₘ—C(═O)N(R⁶)—PEG-C(═O)N(R⁶)—(C₁-C₁₂ alkyldiyl)-C(═O)-Gluc-;
succinimidyl-(CH₂)ₘ—C(═O)N(R⁶)—PEG-O—;
succinimidyl-(CH₂)ₘ—C(═O)N(R⁶)—PEG-O—C(═O)—;
succinimidyl-(CH₂)ₘ—C(═O)N(R⁶)—PEG-C(═O)—;
succinimidyl-(CH₂)ₘ—C(═O)N(R⁶)—PEG-N(R⁵)—;
succinimidyl-(CH₂)ₘ—C(═O)N(R⁶)—PEG-N(R⁵)—C(═O)—;
succinimidyl-(CH₂)ₘ—C(═O)N(R⁶)—PEG-C(═O)—PEP-;
succinimidyl-(CH₂)ₘ—C(═O)N(R⁶)—PEG-SS—(C₁-C₂ alkyldiyl)-OC(═O)—;
-succinimidyl-(CH₂)ₘ—C(═O)—PEP-N(R⁶)—(C₁-C₁₂ alkyldiyl)-;
-succinimidyl-(CH₂)ₘ—C(═O)—PEP-N(R⁶)—(C₁-C₁₂ alkyldiyl)N(R⁶)C(═O)—; and
-succinimidyl-(CH₂)ₘ—C(═O)—PEP-N(R⁶)—(C₁-C₁₂ alkyldiyl)N(R⁶)C(═O)—(C₂-C₅ monoheterocyclyldiyl)-;
R⁶ is independently H or C₁-C₆ alkyl;
PEG has the formula: —(CH₂CH₂O)ₙ—(CH₂)ₘ—; m is an integer from 1 to 5, and n is an integer from 2 to 50;
Gluc has the formula: 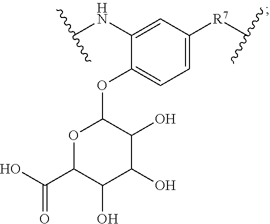
PEP has the formula: 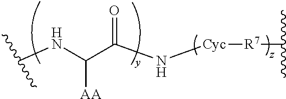
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 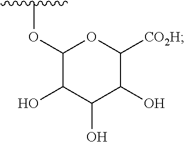 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C═CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)˜H, —OP(O)(OH)₂, —S(O)₂N(CH₃)₂, —SCH₃, —S(O)₂CH₃, and —S(O)₃H.

22. The immunoconjugate of claim 21 wherein the TLR agonist moiety has formula a:

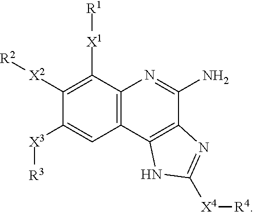

23. The immunoconjugate of claim 21 wherein the TLR agonist moiety has formula b:

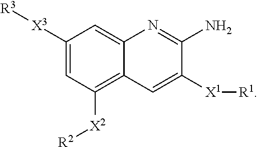

24. The immunoconjugate of claim 21 wherein the TLR agonist moiety has formula c:

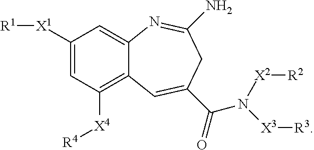

25. The immunoconjugate of claim 21 wherein the TLR agonist moiety has formula d:

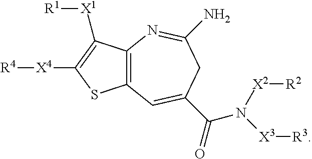

26. The immunoconjugate of claim 21 wherein the TLR agonist moiety has formula e:

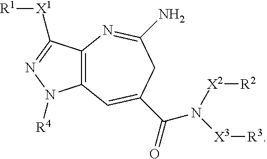

27. The immunoconjugate of claim 21 wherein the TLR agonist moiety has formula f:

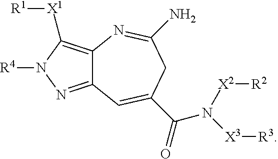

28. (canceled)

29. (canceled)

30. The immunoconjugate of claim 21 wherein X² and X³ are each a bond, and R² and R³ are independently selected from C₁-C₅ alkyl, —O—(C₁-C₁₂ alkyl), —(C₁-C₁₂ alkyldiyl)-OR⁵, —(C₁-C₅ alkyldiyl)-N(R⁵)CO₂R⁵, —(C₁-C₁₂ alkyl)-OC(O)N(R⁵)₂, —O—(C₁-C₁₂ alkyl)-N(R⁵)CO₂R⁵, and —O—(C₁-C₁₂ alkyl)-OC(O)N(R⁵)₂.

31. The immunoconjugate of claim 30 wherein R² is C₁-C₈ alkyl and R³ is —(C₁-C₈ alkyldiyl)-N(R⁵)CO₂R⁴.

32. The immunoconjugate of claim 30 wherein R² is —CH₂CH₂CH₃ and R³ is selected from —CH₂CH₂CH₂NHCO₂(t-Bu), —OCH₂CH₂NHCO₂(cyclobutyl), and —CH₂CH₂CH₂NHCO₂(cyclobutyl).

33. The immunoconjugate of claim 30 wherein R² and R³ are each independently selected from —CH₂CH₂CH₃, —OCH₂CH₃, —OCH₂CF₃, —CH₂CH₂CF₃, —OCH₂CH₂OH, and —CH₂CH₂CH₂OH.

34. The immunoconjugate of claim 30 wherein R² and R³ are each —CH₂CH₂CH₃.

35. The immunoconjugate of claim 30 wherein R² is —CH₂CH₂CH₃ and R³ is —OCH₂CH₃.

36-40. (canceled)

41. The immunoconjugate of claim 21 wherein L is —C(═O)—PEG- or —C(═O)—PEG-C(═O)—.

42. The immunoconjugate of claim 21 wherein L is attached to a cysteine thiol of the antibody.

43. The immunoconjugate of claim 21 wherein for the PEG, m is 1 or 2, and n is an integer from 2 to 10.

44-60. (canceled)

61. A pharmaceutical composition comprising a therapeutically effective amount of an immunoconjugate according to claim 1 and one or more pharmaceutically acceptable diluent, vehicle, carrier or excipient.

62. A method for treating cancer comprising administering a therapeutically effective amount of an immunoconjugate according to claim 1, to a patient in need thereof, wherein the cancer is selected from cervical cancer, endometrial cancer, ovarian cancer, prostate cancer, pancreatic cancer, esophageal cancer, bladder cancer, urinary tract cancer, urothelial carcinoma, lung cancer, non-small cell lung cancer, Merkel cell carcinoma, colon cancer, colorectal cancer, gastric cancer, and breast cancer.

63. The method of claim 62, wherein the cancer is susceptible to a pro-inflammatory response induced by TLR7 and/or TLR8 agonism.

64-68. (canceled)

69. The method of claim 62, wherein the cancer is selected from triple-negative breast cancer, metastatic Merkel cell carcinoma, HER2 overexpressing gastric cancer, and gastroesophageal junction adenocarcinoma.

70. (canceled)

71. (canceled)

72. (canceled)

73. The method of claim 62, wherein the immunoconjugate is administered to the patient intravenously, intratumorally, or subcutaneously.

74. The method of claim 62, wherein the immunoconjugate is administered to the patient at a dose of about 0.01-20 mg per kg of body weight.

75. (canceled)

76. A TLR agonist-linker (TLR-L) compound selected from the group consisting of: 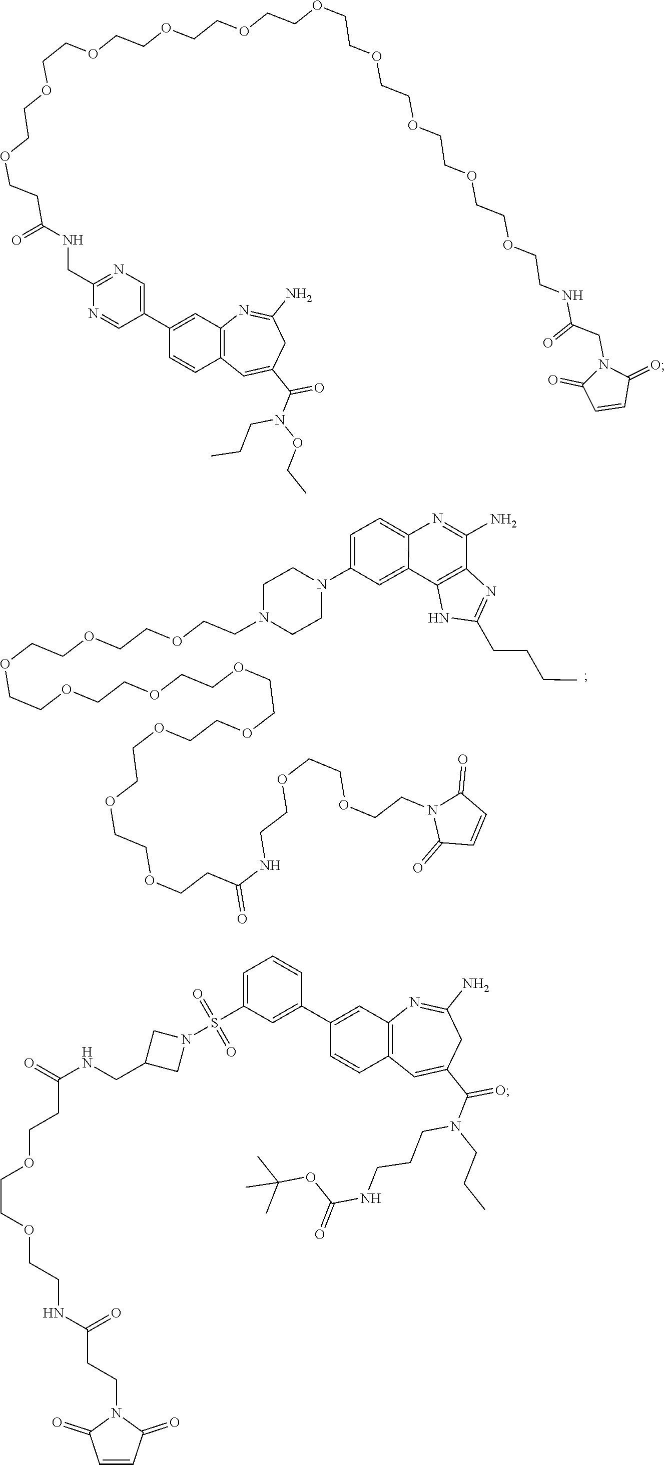 [SMILES: CCCN(OCC)C(=O)C1=Cc2ccc(-c3cnc(CNC(=O)CCOCCOCCOCCOCCOCCOCCOCCOCCOCCOCCNC(=O)CN4C(=O)C=CC4=O)nc3)cc2N=C(N)C1 ; CCCCc1nc2c(N)nc3ccc(N4CCN(CCOCCOCCOCCOCCOCCOCCOCCOCCOCCOCCC(=O)NCCOCCOCCN5C(=O)C=CC5=O)CC4)cc3c2[nH]1 ; CCCN(CCCNC(=O)OC(C)(C)C)C(=O)C1=Cc2ccc(-c3cccc(S(=O)(=O)N4CC(CNC(=O)CCOCCOCCNC(=O)CCN5C(=O)C=CC5=O)C4)c3)cc2N=C(N)C1] 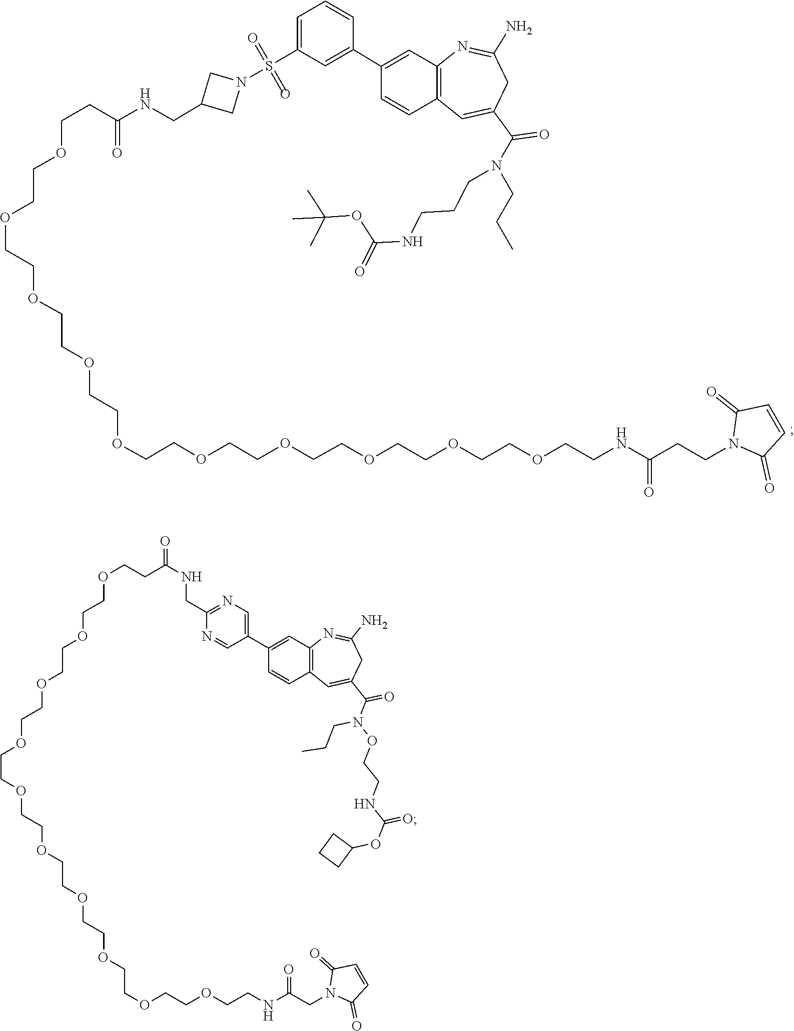 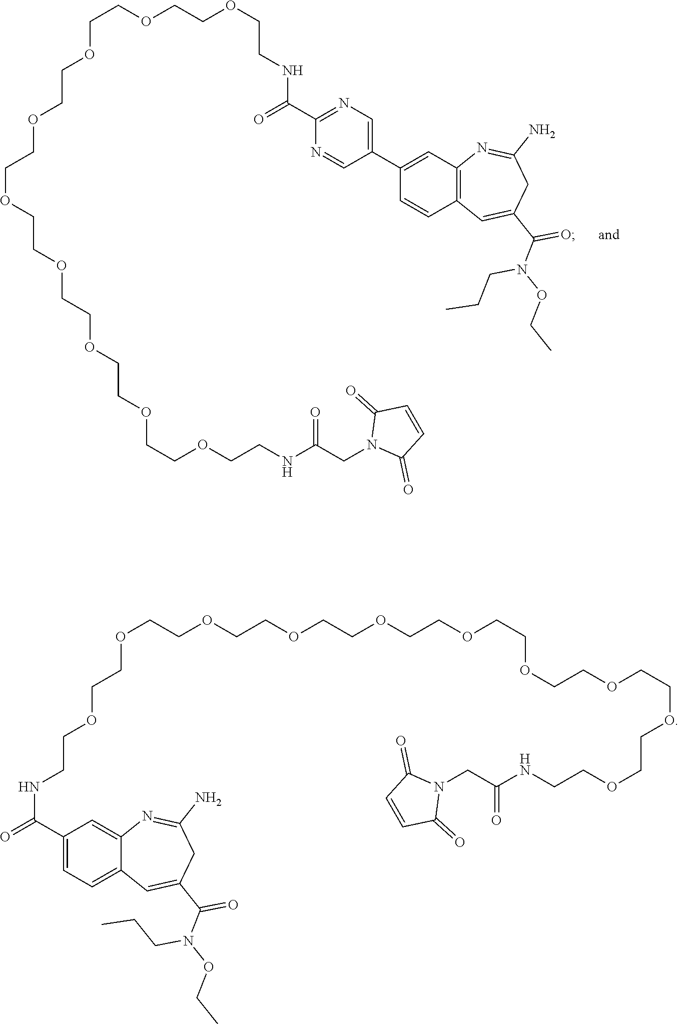 [SMILES: CCCN(OCC)C(=O)C1=Cc2ccc(-c3cnc(C(=O)NCCOCCOCCOCCOCCOCCOCCOCCOCCOCCNC(=O)CN4C(=O)C=CC4=O)nc3)cc2N=C(N)C1 ; "and" ; CCCN(OCC)C(=O)C1=Cc2ccc(C(=O)NCCOCCOCCOCCOCCOCCOCCOCCOCCOCCOCCNC(=O)CN3C(=O)C=CC3=O)cc2N=C(N)C1]
